(19) United States
(12) Patent Application Publication
(10) Pub. No.: US 2019/0290892 A1
(43) Pub. Date: Sep. 26, 2019

(54) METHODS OF DETERMINING DOSING OF A THERAPEUTIC AGENT BASED ON MEASURED LEVELS OF A METABOLITE

(71) Applicant: Clear Creek Bio, Inc., Cambridge, MA (US)

(72) Inventors: Vikram S. Kumar, Boston, MA (US); David P. Hesson, Malvern, PA (US)

(21) Appl. No.: 16/364,423

(22) Filed: Mar. 26, 2019

Related U.S. Application Data
(60) Provisional application No. 62/682,419, filed on Jun. 8, 2018.
(60) Provisional application No. 62/655,407, filed on Apr. 10, 2018.
(60) Provisional application No. 62/648,320, filed on Mar. 26, 2018.

(51) Int. Cl. A61M 31/00; A61K 35/00; G01N 33/573; G01N 33/535; G01N 33/574; G01R 33/60

(52) U.S. Cl. CPC A61M 31/002; C12Y 401/01023; G01N 33/573; G01N 33/535; G01N 33/57426; G01N 33/57434; G01R 33/60; A61M 2205/0227; A61M 2205/3303; A61M 2205/3327; A61M 2205/702; C12Y 201/03002; C12Y 204/0201; C12Y 103/01014; A61K 35/00

(57) ABSTRACT

The invention provides methods of determining a therapeutically effective dose of an agent that targets a metabolic pathway based on measured levels of a metabolite in the pathway. The methods, which may include providing the agent in a therapeutically effective dose, are useful for treating disorders, such as cancer, in a subject. The invention also provides methods for assessing the impact of a therapeutic agent on a tumor in a subject by monitoring, in real time, metabolism of a molecule in the tumor, oxygenation of the tumor, or both. The invention further provides devices that determine a therapeutically effective dose of an agent that targets a metabolic pathway based on measured levels of a metabolite in the pathway and notify a subject to administer the dose.

### CROSS-REFERENCE TO RELATED APPLICATIONS

This application claims the benefit of, and priority to, U.S. Provisional Application No. 62/648,320, filed Mar. 26, 2018; U.S. Provisional Application No. 62/655,407, filed Apr. 10, 2018; and U.S. Provisional Application No. 62/682,419, filed Jun. 8, 2018, the contents of each of which are incorporated herein by reference.

### FIELD OF THE INVENTION

The invention relates generally to methods of determining dosing of a therapeutic agent based on measured levels of a metabolite in a pathway that is targeted by the therapeutic agent.

### BACKGROUND

The use of drugs to treat virtually any type of condition is a primary tool of modern medicine. Despite the ubiquity of drug therapies, however, our ability to evaluate whether an active pharmaceutical ingredient (API) is exerting the desired effect in an individual patient is highly limited.

One shortcoming of existing methods is that they rely on drug dosing schedules that may not be suited for a given individual. Dosing is often based on pharmacokinetic studies that examine the rate of metabolism and elimination of the drug from the body and yield data that represent the average rate of drug metabolism from a population of patients. However, individual patients differ vastly in how they metabolize certain drugs, and those differences may be critical for drugs that have a narrow therapeutic window, i.e., a restricted range between the minimum dosage at which a drug is effective and the dosage at which it is toxic. In such cases, administration of the drug must be accompanied by ongoing measurement, i.e., monitoring, of one or more physiological parameters in the patient's body and adjustment of the drug dosage based on the measured values.

A complicating problem is that current methods of monitoring are flawed. The physiological parameters that are measured in the patient's body, such as the level of the API or a metabolite of the API, do not necessarily reflect whether a particular API has engaged its intended target. Because the kinetics of functional interaction between the API and its target often differ from the measured parameters, dosing schedules based on those measurements may lead to excessive side effects or fail to control the primary condition. Consequently, current methods for obtaining information on drug efficacy in real time and using that information to provide patient-tailored drug dosing are inadequate, and millions of individuals continue to suffer from improperly medicated ailments or unnecessary drug side effects.

### SUMMARY

The invention provides methods and devices that allow physicians to determine, optionally in real time, a therapeutically effective dose of a drug for an individual patient by examining levels of a metabolite in a pathway targeted by the drug. In particular embodiments, the effectiveness of a drug containing an enzyme inhibitor is assessed by analyzing the level of the enzyme's substrate in a sample obtained from the patient. Because activity of the enzyme can be inferred from substrate levels, engagement of the API with its target can be evaluated, optionally in real time, and drug dosage can be adjusted accordingly. Thus, compared to prior methods that rely on levels of the API or a metabolite of the API to calibrate drug dosage, the methods of the invention yield dosing schedules that afford better control of a variety of diseases, disorders, and conditions and decrease the risk harmful drug side effects. The invention also provides devices that notify patients, in real time, of recommended adjustments to their dosing regimens based on measured levels of a metabolite in the pathway targeted by the drug.

Because the methods permit real-time adjustment of drug dosage to optimize therapeutic effectiveness, they are useful for treatment of various diseases, such as cancer. For example, control of dihydroorotate dehydrogenase (DHODH) in acute myeloid leukemia could selectively starve leukemia cells, so the DHODH inhibitor brequinar has potential as an anti-cancer agent. However, achieving a therapeutically effective dosing regimen of brequinar is problematic: when the drug is administered frequently, e.g., daily, it causes toxic side effects, and when it is administered too infrequently, e.g., biweekly or on a schedule that requires extended “off” periods between doses, it has no therapeutic benefit. Methods of the invention solve this problem by monitoring levels of dihydroorotate (DHO), the substrate for DHODH, in the patient's body to determine the frequency and dose for administration of brequinar. Consequently, the invention unlocks the therapeutic potential of brequinar and other drugs that have narrow therapeutic windows or high interindividual variability.

The invention also provides methods of evaluating the effectiveness of anti-cancer agents by assessing their effects on tumors, optionally in real time. The methods involve analyzing properties of a tumor in the body, such as the flux or single point level of a nutrient, substrate, or metabolite in the tumor or the level of oxygenation of the tumor. By monitoring these properties in a patient who has been given a therapeutic agent, the impact of the drug on the tumor can be gauged, and the dosing regimen can be adjusted accordingly.

In an aspect, the invention provides methods for determining a therapeutically effective dose of an agent to treat a disorder in a subject. The methods include receiving information regarding a measured level of a metabolite in a metabolic pathway in a sample from a subject having a disorder, comparing the received information to a reference that provides an association of a measured level of the metabolite with a recommended dosage adjustment of an agent, and determining, based on the comparing step, a dosage of the agent that results in the level of the metabolite being raised or maintained above a threshold level. The threshold level is indicative that a sufficient amount of the agent is present in the subject to sufficiently alter the metabolic pathway to ameliorate, reduce, or eliminate at least one sign or symptom of the disorder.

In an aspect, the invention provides methods for determining a therapeutically effective dose of an agent to be provided to a subject to treat a disorder. The methods include determining a therapeutically effective dose of an agent based on a measured level of a metabolite in a nucleotide synthesis pathway in a sample from a subject. The therapeutically effective dose of the agent inhibits an enzyme within the nucleotide synthesis pathway to an extent that at least one sign or symptom of the disorder is ameliorated, reduced, or eliminated.

The recommend dosage adjustment may include a change in the dosage. For example, the recommend dosage adjustment may include an increase of the dosage by a certain value, a decrease of the dosage by a certain value, or no adjustment to the dosage. The recommended dosage adjustment may include a change in the schedule of providing the dose. For example, the recommended dosage adjustment may include an increase in the interval between doses, a decrease in the interval between doses, or no change in the interval between doses.

The agent may be any therapeutic agent. For example, the agent may be PALA (N-phosphoacetyl-L-aspartate), brequinar, pyrazofurin, brequinar, a brequinar analog, a brequinar derivative, a brequinar prodrug, a micellar formulation of brequinar, or a brequinar salt. The agent may inhibit an enzyme in the metabolic pathway. For example, the agent may inhibit aspartate transcarbamoylase, dihydrooratase, dihydroorotate dehydrogenase, orotidine 5′-monophosphate (OMP) decarboxylase, or orotate phosphoribosyl transferase.

The metabolite may be a substrate or product of an enzyme in the metabolic pathway targeted by the drug. The metabolic pathway may be a nucleotide synthesis pathway, such as a pyrimidine synthesis pathway or a purine synthesis pathway. The metabolite may be an intermediate in a nucleotide synthesis pathway. For example, the metabolite may be N-carbamoylaspartate, dihydroorotate, orotate, orotidine 5′-monophosphate (OMP), or uridine monophoshpate (UMP).

The disorder may be any disorder, disease, or condition for which altering the activity of a metabolic pathway can be of therapeutic benefit. The disorder may be one in which inhibiting an enzyme in a metabolic pathway is of therapeutic benefit. The disorder may be cancer or an autoimmune disorder. The cancer may be leukemia, such as acute myeloid leukemia (AML), PTEN null prostate cancer, lung cancer, such as small cell lung cancer and non-small cell lung cancer, triple negative breast cancer (TNBC), glioma, multiple myeloma, acute lymphoblastic leukemia (ALL), neuroblastoma, or adult T cell leukemia/lymphoma (ATLL). The autoimmune disorder may be arthritis or multiple sclerosis.

The methods may include additional steps. For example, the method may include measuring the level of the metabolite in a sample obtained from the subject or providing the agent to the subject at the determined dose.

The sample may be a body fluid sample. For example, the body fluid may be plasma, blood, serum, urine, sweat, saliva, interstitial fluid, feces, or phlegm

In an aspect, the invention provides methods for assessing the impact of a therapeutic agent on a tumor in real time. The methods include monitoring in real time a molecule that is associated with a metabolic pathway as the molecule moves through the metabolic pathway in a tumor in a subject and assessing the impact on the tumor of a therapeutic agent that has been administered to a subject based on results of the monitoring.

In an aspect, the invention provides methods for assessing the impact of a therapeutic agent on tumor in real time. The methods include monitoring in real time an oxygenation level in a tumor and assessing the impact on the tumor of a therapeutic agent that has been administered to a subject based on results of the monitoring step.

In an aspect, the invention provides methods for assessing the impact of a therapeutic agent on tumor in real time. The methods include monitoring in real time a molecule that is associated with a metabolic pathway as the molecule moves through the metabolic pathway in a tumor in a subject, monitoring in real time an oxygenation level in a tumor, and assessing the impact on the tumor of a therapeutic agent that has been administered to a subject based on results of the monitoring step.

The monitoring may include any suitable method. For example, monitoring the molecule in the tumor may include the use of hyperpolarization magnetic resonance imaging, and monitoring the oxygenation level of the tumor may include electron paramagnetic resonance (EPR) imaging.

The molecule may be a carbon molecule. The molecule may be or become associated with a metabolite in a metabolic pathway. For example, the metabolite may be N-carbamoylaspartate, dihydroorotate, orotate, orotidine 5′-monophosphate (OMP), or uridine monophoshpate (UMP).

The metabolic pathway may be any metabolic pathway, as described above. For example, the metabolic pathway may a nucleotide synthesis pathway.

The agent may be any therapeutic agent, as described above.

The methods may include quantifying the molecule. Quantifying the molecule may quantify the level of a metabolite in a metabolic pathway, such as dihydroorotate or orotate.

The methods may include determining, based on the levels of a metabolite, such as dihydroorotate or orotate, a dose of the therapeutic agent that is sufficient to inhibit an enzyme within the metabolic pathway, such as a nucleotide synthesis pathway, to an extent that at least one sign or symptom of the disorder is ameliorated, reduced, or eliminated.

The methods may include repeating one or more of the monitoring, assessing, and determining steps at different points in time. The methods may include adjusting the dose of the therapeutic agent based on results of the method from a subsequent point in time.

In another aspect, the invention provides devices for notifying a subject having a disorder that a dose of therapeutic agent that targets a metabolic pathway should be administered to the subject. The devices include a processor coupled to a memory unit that causes the processor to receive data that includes a dose of the therapeutic agent and the time the dose was received by the subject, generate a reminder that includes the time the next dose should be administered to the subject, and output the reminder to the subject. The time for administering the next dose to the subject is based on a relationship between the dose of the therapeutic agent and a threshold level of the metabolite, and administration of the next dose raises or maintains a level of the metabolite above the threshold. The threshold level is indicative that a sufficient amount of the agent is present in the subject to sufficiently alter the metabolic pathway to ameliorate, reduce, or eliminate at least one sign or symptom of the disorder.

The reminder may be any type of notification that can be perceived by a human. For example, the reminder may be an audible signal, a visual signal, a tactile signal, a vibration, or a combination thereof.

The reminder may be outputted to a component of the device. Additionally, or alternatively, the reminder may be outputted to a remote device.

Each of the time when the dose was received by the subject and the time when the next dose should be administered may include any temporal component. For example, each of the times may include a date, day of the week, hour, minute, second, or time zone.

The device may store information related to the time when the dose was received by the subject, the time when the next dose should be administered, or both. The information may be stored in the memory unit.

The process may perform calculations on the stored information. For example, the process may determine whether intervals between time points, such as times when individual doses are received by the subject, change over time. The processor may determine that the subject has developed resistance or is developing resistance to a therapeutic agent based on the stored information. For example, the processor may determine that the subject has developed resistance or is developing resistance to a therapeutic agent based on a change in the intervals over time, such as a decrease in the intervals over time, a change in the dose over time, such as an increase in the dose over time, or both.

The processor may output a recommendation for adjusting a therapeutic course for the subject. For example, the recommendation may include altering, e.g., increasing or decreasing, a dose, or altering, e.g., increasing or decreasing, an interval between doses. The recommendation may include administering a second therapeutic agent in addition to the first therapeutic agent. The recommendation may include a dose for administration of the second therapeutic agent, a time for administration of the second therapeutic agent, or both.

The processor may output stored information to a physician. For example, the processor may output information on doses of the therapeutic agent received by the subject, time points when the therapeutic agent was received by the subject, or both to a physician. The stored information may enable the physician to determine that the subject has developed or is developing resistance to the therapeutic agent. For example, the information may enable the physician to determine that the subject has developed resistance or is developing resistance to a therapeutic agent based on a change in the intervals of receiving the therapeutic agent over time, such as a decrease in the intervals over time, a change in the dose of the therapeutic agent over time, such as an increase in the dose over time, or both. The stored information may enable the physician to adjust the therapeutic course for the subject. For example, the stored information may enable the physician to alter the dose of the therapeutic agent, the time when the therapeutic agent should be administered, or both.

### BRIEF DESCRIPTION OF THE DRAWINGS

FIG. 1 is a series of graphs showing levels of brequinar and DHO in three patients that have received a single dose of brequinar according to the same dosing regimen.

FIG. 2 is a series of graphs showing levels of brequinar and DHO in three patients that have received multiple doses of brequinar according to the same dosing regimen.

FIG. 3 is a flow chart illustrating an example of determining dose of a DHODH inhibitor for a patient according to an embodiment of the invention.

FIG. 4 is a scatter plot illustrating the concentration of brequinar in subject plasma over time when administered twice weekly.

FIG. 5 is a scatter plot illustrating the bioavailability of an IV formulation of brequinar as compared to an oral dosage form.

FIG. 6 is a scatter plot illustrating the concentration of brequinar in mice at a dose of 50 mg/kg over time.

FIG. 7 is a scatter plot illustrating the baseline DHO levels in random cancer patients and healthy patients, as reported in Table 5.

FIG. 8 is a scatter plot illustrating the concentrations of pyrazofurin and orotate in murine plasma over time when pyrazofurin is administered as a single dose (20 mg/kg).

FIG. 9 is a scatter plot illustrating the concentrations of pyrazofurin and orotate in murine plasma over time when pyrazofurin is administered as a single dose (20 mg/kg) on a log scale.

FIG. 10 is a graph showing the therapeutic benefit of a drug that targets a metabolic pathway as a function of levels of a metabolite that is an intermediate in the pathway.

### DETAILED DESCRIPTION

The invention provides methods that allow real-time determination of therapeutically effective dosing regimens of drugs that include an enzyme inhibitor as an active pharmaceutical ingredient (API). The methods are based on the insight that the extent to which the target enzyme is engaged by the inhibitor can be evaluated based on measured levels of a metabolite in a pathway in which the enzyme functions. In particular, target engagement can be assessed from levels of a substrate of the enzyme. From the measured level of a metabolite in a sample obtained from a patient, the methods allow a physician to determine an appropriate amount of drug that contains an enzyme inhibitor to administer to the patient to alleviate a sign or symptom of a disorder and minimize undesirable side effects of the drug.

The methods of the invention greatly improve the utility of drugs that have large interpatient variability in drug metabolism or a narrow therapeutic window, i.e., drugs for which the range between doses necessary to achieve therapeutic effect and doses that cause toxicity is small. Administration of such drugs requires precise dosing and typically includes monitoring of their effects on patients. Monitoring often involves measurement of the level of the API or a metabolic product of the API in the patient's body. However, patients vary widely in their ability to metabolize drugs and in how drugs affect targets in their bodies, so analysis of the API or a metabolic product thereof provides an incomplete readout of the efficacy of a given drug in an individual patient. The invention overcomes this limitation by using levels of a metabolite in an enzymatic pathway as a metric of engagement of the API with its target enzyme. Whereas patient variability makes drug efficacy difficult to ascertain precisely from levels of an API or a metabolic product of the API, levels of a metabolite in the pathway of the API's target are universal indicators of target engagement. Thus, because the dosing regimen is determined based on levels of the metabolite rather than levels of the drug, the methods of the invention afford greater precision in the dosage and timing of drug administration. Consequently, the methods enable the safe and effective treatment of a variety of conditions using therapeutic agents that are ineffective or too dangerous under prior methods.

According to methods of the invention, drug dosage is determined based on real-time measured levels of a metabolite in a patient. The levels may be measured in a sample, such as plasma sample, obtained from a patient. In such embodiments, the methods permit rapid, convenient monitoring of patients. Alternatively, levels of the metabolite may be measured in a tumor in vivo. Thus, the invention also provides methods that allow direct, real-time assessment of the effect of a therapeutic agent on a tumor in the patient's body.

The invention further provides devices, such as wearable electronic devices, that provide reminders to a patient regarding drug dosing, such as the dosage of a drug or time for administration. The notifications that the devices provide are based on one or more measured values of a metabolite in a sample obtained from the patient. Thus, the devices incorporate the aforementioned advantages of the methods provided herein.

### Metabolites as Indicators of Target Engagement

Methods of the invention include determining the dosage of a drug based on a measured level of a metabolite in a sample obtained from a subject. The metabolite may be any molecule that provides an indication of target engagement by the API of the drug. In embodiments of the invention, the API is an inhibitor of an enzyme in a metabolic pathway, and the metabolite is an intermediate the pathway. Preferably, the metabolite the API is an inhibitor of an enzyme in a metabolic pathway, and the metabolite is a substrate of the enzyme.

Nucleotide synthesis pathways are of particular therapeutic interest. The high proliferation rate of cancer cells often places increased demand on nucleotide synthesis pathways. Consequently, enzymes that function in such pathways are useful targets for antineoplastic drugs. Specifically, drugs that inhibit enzymes require for nucleotide synthesis have been investigated for treating cancer. Therefore, levels of metabolites in nucleotide synthesis pathways are useful for evaluating the extent to which the APIs in such drugs are engaging their targets in vivo.

Pyrimidine biosynthesis involves a sequence of step enzymatic reactions that result in the conversion of glutamine to uridine monophosphate as shown below:

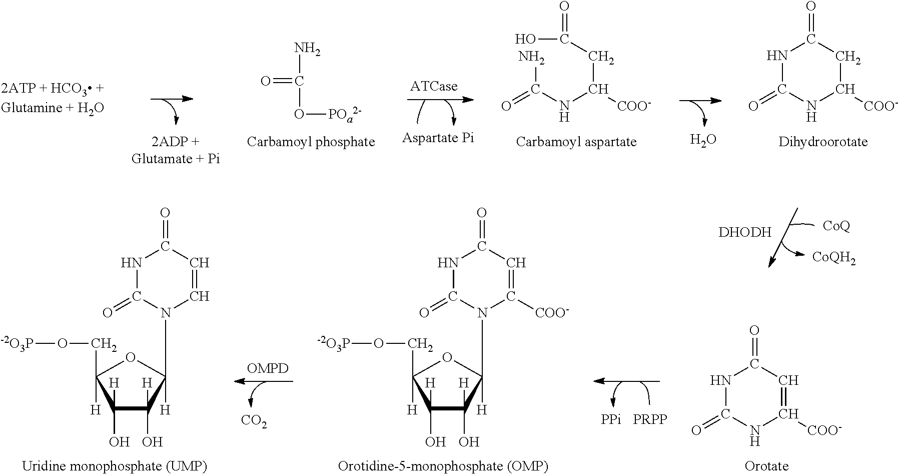

Several of the enzymes in the pyridine synthesis pathway are targets of drugs or drug candidates. For example, inhibitors of the following enzymes have been investigated as therapeutic agents: aspartate carbamoyltransferase (also known as aspartate transcarbamoylase or ATCase), which catalyzes the conversion of carbamoyl phosphate to carbamoyl aspartate; dihydroorotate dehydrogenase (DHODH), which catalyzes conversion of dihydroorotate (DHO) to orotate; and OMP decarboxylase (OMPD), which catalyzes conversion of orotidine monophosphate (OMP) to uridine monophosphate (UMP).

One element of the invention is recognition of the utility of DHO as an indicator of target engagement by DHODH inhibitors. One advantage of DHO is that cell membranes are permeable to the molecule. DHODH is localized to the mitochondrial inner membrane within cells, making direct measurement of enzyme activity difficult. However, DHO, which accumulates when DHODH is inhibited, diffuses out of cells and into the blood, which can be easily sampled. Another insight of the invention is that DHO is sufficiently stable that levels of the metabolite can be measured reliably. Previously, DHO was considered too unstable at ambient temperatures to be quantified accurately and was thus deemed unsuitable as an indicator of DHODH inhibition. However, the methods provided herein permit detection of DHO in plasma samples. Thus, by analyzing levels of DHO in blood or blood products, one can readily assess target engagement of a DHODH inhibitor.

In an analogous manner, orotate and OMP can serve as indicators for target engagement of OMP decarboxylase inhibitors. For example, inhibition of OMP decarboxylase leads to increased plasma levels of orotate, so measurement of plasma orotate levels is useful for assessing the effect of agents that target OMP decarboxylase.

The methods of the invention are applicable for therapeutic agents that regulate the activity of other metabolic pathways as well. Examples of such pathways include the purine synthesis pathway, which is targeted by methotrexate and 6-mercaptopurine and in which an enzyme inosine-5′-monophosphate dehydrogenase (IMPDH) may be targeted; the anandamide degradation pathway, including the enzyme fatty acid amide hydrolase, which is targeted by a variety of inhibitors and activators; and glycolysis, the citric acid cycle, and the balance between the two, which are targeted by various drug candidates; the pentose phosphate pathway; and the beta-oxidation pathway.

### Measuring the Level of a Metabolite in a Sample

Methods of the invention include analysis of a measured level of metabolite in a sample. The methods may include measurement of the metabolite.

In some embodiments, the metabolite is measured by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), hydrophilic interaction chromatography (HILIC), ultra-performance liquid chromatography (UPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi: 10.1002/elps.200500398; Kolch et al., Capillary electrophoresis-mass spectrometry as a powerful tool in clinical diagnosis and biomarker discovery, Mass Spectrom Rev. 24 (6): 959-77. (2005) doi:10.1002/mas.20051; Kanu et al., Ion mobility-mass spectrometry, Journal of Mass Spectrometry, 43 (1): 1-22 (2008) doi: 10.1002/jms. 1383, the contents of which are incorporated herein by reference.

A sample may be obtained from any organ or tissue in the individual to be tested, provided that the sample is obtained in a liquid form or can be pre-treated to take a liquid form. For example and without limitation, the sample may be a blood sample, a urine sample, a serum sample, a semen sample, a sputum sample, a lymphatic fluid sample, a cerebrospinal fluid sample, a plasma sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, a phlegm sample, a saliva sample, a sweat sample, or a combination of such samples. The sample may also be a solid or semi-solid sample, such as a tissue sample, feces sample, or stool sample, that has been treated to take a liquid form by, for example, homogenization, sonication, pipette trituration, cell lysis etc. For the methods described herein, it is preferred that a sample is from plasma, serum, whole blood, or sputum.

The sample may be kept in a temperature-controlled environment to preserve the stability of the metabolite. For example, DHO is more stable at lower temperatures, and the increased stability facilitates analysis of this metabolite from samples. Thus, samples may be stored at 4° C., −20° C., or −80° C.

In some embodiments, a sample is treated to remove cells or other biological particulates. Methods for removing cells from a blood or other sample are well known in the art and may include e.g., centrifugation, sedimentation, ultrafiltration, immune selection, etc.

The subject may be an animal (such as a mammal, such as a human). The subject may be a pediatric, a newborn, a neonate, an infant, a child, an adolescent, a pre-teen, a teenager, an adult, or an elderly patient. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The sample may be obtained from an individual before or after administration to the subject of an agent that alters activity of a metabolic pathway, such as inhibitor of an enzyme in the pathway. For example, the sample may be obtained 1 hour, 2 hours, 4 hours, 6 hours, 8 hours, 12 hours, 24 hours, 36 hours, 2 days, 3 days, 4 days, 5 days, 6 days, 7 days or more before administration of an agent, or it may be obtained 1 hour, 2 hours, 4 hours, 6 hours, 8 hours, 12 hours, 24 hours, 36 hours, 2 days, 3 days, 4 days, 5 days, 6 days, 7 days or more after administration of an agent.

### Determining Dosing Regimens

Methods of the invention include determining a dosing regimen of an agent that alters a metabolic pathway, such as an inhibitor of an enzyme in the pathway, for a subject. The dosing regimen may include a dose, i.e., an amount, of the agent that should be administered. The dosing regimen may include a time point for administration of a dose of the agent to the subject. Because the dosing regimen is based on one or more measured levels of a metabolite in a sample obtained from the subject, the dosing regimen is tailored to an individual subject, e.g., a patient. Consequently, the methods of the invention provide customized dosing regimens that account for variability in pharmacokinetic properties, i.e., metabolism of the API by the subject, and pharmacodynamics properties, effect of the API on its target, among individuals.

The dosing regimen may be determined by comparing a measured level of a metabolite in a sample obtained from a subject to a reference that provides an association between the measured level and a recommended dosage adjustment of the agent. For example, the reference may provide a relationship between administration of the agent and levels of the metabolite in the subject. The relationship can be empirically determined from a known dose and time of administration of the agent and measured levels of the metabolite at one or more subsequent time points. The reference may include a relationship between measured levels of the agent or a metabolic product of the agent and measured levels of the metabolite.

From the comparison between the measured level of the metabolite and the reference, a dosing regimen may then be determined. The dosing regimen may include a dosage of the agent, a time for administration of the dosage, or both. The dosing regimen may be determined de novo, or it may comprise an adjustment to a previous dosing regimen, such as an adjustment in the dosage, the interval between administration of dosages, or both.

The dosing regimen is designed to deliver the agent to the subject in an amount that achieves a therapeutic effect. The therapeutic effect may be a sign or symptom of a disease, disorder, or condition. The therapeutic effect may be inhibition of an enzyme in the metabolic pathway, or it may be a change in an indicator of inhibition of an enzyme in a metabolic pathway. The indicator may be a metabolite in the pathway, and the therapeutic effect may be an increase or decrease in levels of the metabolite. The therapeutic effect may be a decrease in number of cancer cells, a decrease in proliferation of cancer cells, an increase in differentiation of pre-cancerous cells, such as myeloblasts, complete remission of cancer, complete remission with incomplete hematologic recovery, morphologic leukemia-free state, or partial remission. Increased differentiation of myeloblasts may be assessed by one or more of expression of CD14, expression of CD11b, nuclear morphology, and cytoplasmic granules.

The dosing regimen may ensure that levels of a metabolite are raised or maintained a minimum threshold required to achieve a certain effect. For example, the dosing regimen may raise or maintain levels of the metabolite above a threshold level in the subject for a certain time period. The time period may include a minimum, a maximum, or both. For example, the dosing regimen may raise or maintain levels of the metabolite above the threshold level for at least 6 hours, 12, hours, 24 hours, at least 48 hours, at least 60 hours, at least 72 hours, at least 84 hours, at least 96 hours, at least 5 days, at least 6 days, at least 7 days, at least 10 days, at least 2 weeks, or more. The dosing regimen may raise or maintain levels of the metabolite above the threshold level for not more than 24 hours, not more than 36 hours, not more than 48 hours, not more than 60 hours, not more than 72 hours, not more than 84 hours, not more than 96 hours, not more than 5 days, not more than 6 days, not more than 7 days, not more than 10 days, or not more than 2 weeks. The dosing regimen may raise or maintain levels of the metabolite above the threshold level for at least 72 hours but not more than 96 hours, for at least 72 hours but not more than 5 days, for at least 72 hours but not more than 6 days, for at least 72 hours but not more than 7 days, for at least 96 hours but not more than 7 days.

The dosing regimen may ensure that levels of a metabolite do not exceed or are maintained below a maximum threshold that is associated with toxicity. Levels of the metabolite above a maximum threshold may indicate that the agent is causing or is likely to cause an adverse event in the subject. For example and without limitation, adverse events include abdominal pain, anemia, anorexia, blood disorders, constipation, diarrhea, dyspepsia, fatigue, fever, granulocytopenia, headache, infection, leukopenia, mucositis, nausea, pain at the injection site, phlebitis, photosensitivity, rash, somnolence, stomatitis, thrombocytopenia, and vomiting.

The dosing regimen may include a time point for administration of one or more subsequent doses to raise or maintain levels of the metabolite above a threshold level for a certain time period. The time point for administration of a subsequent dose may be relative to an earlier time point. For example, the time point for administration of a subsequent dose may be relative to a time point when a previous dose was administered or a time point when a sample was obtained from a subject.

The dosing regimen may include a schedule for administration of doses. For example, doses may be administered at regular intervals, such as every 24 hours, every 36 hours, every 48 hours, every 60 hours, every 72 hours, every 84 hours, every 96 hours, every 5 days, every 6 days, every week, every 2 weeks, every 3 weeks, or every 4 weeks. Alternatively, doses may be administered according to a schedule that does not require precisely regular intervals. For example, doses may be administered once per week, twice per week, three times per week, four times per week, once per month, twice per month, three times per month, four times per month, five times per month, or six times per month.

For example and without limitation, a dosing regimen for administration of a therapeutic agent, such brequinar, e.g., brequinar sodium, to a human subject may be as follows: 100 mg/m², administered intravenously twice weekly; 125 mg/m², administered intravenously twice weekly; 150 mg/m², administered intravenously twice weekly; 200 mg/m², administered intravenously twice weekly; 250 mg/m², administered intravenously twice weekly; 275 mg/m², administered intravenously twice weekly; 300 mg/m², administered intravenously twice weekly; 350 mg/m², administered intravenously twice weekly; 400 mg/m², administered intravenously twice weekly; 425 mg/m², administered intravenously twice weekly; 450 mg/m², administered intravenously twice weekly; 500 mg/m², administered intravenously twice weekly; 550 mg/m², administered intravenously twice weekly; 600 mg/m², administered intravenously twice weekly; 650 mg/m², administered intravenously twice weekly; 700 mg/m², administered intravenously twice weekly; 750 mg/m², administered intravenously twice weekly; 800 mg/m², administered intravenously twice weekly; 100 mg/m², administered intravenously every 72 hours; 125 mg/m², administered intravenously every 72 hours; 150 mg/m², administered intravenously every 72 hours; 200 mg/m², administered intravenously every 72 hours; 250 mg/m², administered intravenously every 72 hours; 275 mg/m², administered intravenously every 72 hours; 300 mg/m², administered intravenously every 72 hours; 350 mg/m², administered intravenously every 72 hours; 400 mg/m², administered intravenously every 72 hours; 425 mg/m², administered intravenously every 72 hours; 450 mg/m², administered intravenously every 72 hours; 500 mg/m², administered intravenously every 72 hours; 550 mg/m², administered intravenously every 72 hours; 600 mg/m², administered intravenously every 72 hours; 650 mg/m², administered intravenously every 72 hours; 700 mg/m², administered intravenously every 72 hours; 750 mg/m², administered intravenously every 72 hours; 800 mg/m², administered intravenously every 72 hours; 100 mg/m², administered intravenously every 84 hours; 125 mg/m², administered intravenously every 84 hours; 150 mg/m², administered intravenously every 84 hours; 200 mg/m², administered intravenously every 84 hours; 250 mg/m², administered intravenously every 84 hours; 275 mg/m², administered intravenously every 84 hours; 300 mg/m², administered intravenously every 84 hours; 350 mg/m², administered intravenously every 84 hours; 400 mg/m², administered intravenously every 84 hours; 425 mg/m², administered intravenously every 84 hours; 450 mg/m², administered intravenously every 84 hours; 500 mg/m², administered intravenously every 84 hours; 550 mg/m², administered intravenously every 84 hours; 600 mg/m², administered intravenously every 84 hours; 650 mg/m², administered intravenously every 84 hours; 700 mg/m², administered intravenously every 84 hours; 750 mg/m², administered intravenously every 84 hours; 800 mg/m², administered intravenously every 84 hours; 100 mg/m², administered intravenously every 96 hours; 125 mg/m², administered intravenously every 96 hours; 150 mg/m², administered intravenously every 96 hours; 200 mg/m², administered intravenously every 96 hours; 250 mg/m², administered intravenously every 96 hours; 275 mg/m², administered intravenously every 96 hours; 300 mg/m², administered intravenously every 96 hours; 350 mg/m², administered intravenously every 96 hours; 400 mg/m², administered intravenously every 96 hours; 425 mg/m², administered intravenously every 96 hours; 450 mg/m², administered intravenously every 96 hours; 500 mg/m², administered intravenously every 96 hours; 550 mg/m², administered intravenously every 96 hours; 600 mg/m², administered intravenously every 96 hours; 650 mg/m², administered intravenously every 96 hours; 700 mg/m², administered intravenously every 96 hours; 750 mg/m², administered intravenously every 96 hours; 800 mg/m², administered intravenously every 96 hours; 100 mg/m², administered orally twice weekly; 125 mg/m², administered orally twice weekly; 150 mg/m², administered orally twice weekly; 200 mg/m², administered orally twice weekly; 250 mg/m², administered orally twice weekly; 275 mg/m², administered orally twice weekly; 300 mg/m², administered orally twice weekly; 350 mg/m², administered orally twice weekly; 400 mg/m², administered orally twice weekly; 425 mg/m², administered orally twice weekly; 450 mg/m², administered orally twice weekly; 500 mg/m², administered orally twice weekly; 550 mg/m², administered orally twice weekly; 600 mg/m², administered orally twice weekly; 650 mg/m², administered orally twice weekly; 700 mg/m², administered orally twice weekly; 750 mg/m², administered orally twice weekly; 800 mg/m², administered orally twice weekly; 100 mg/m², administered orally every 72 hours; 125 mg/m², administered orally every 72 hours; 150 mg/m², administered orally every 72 hours; 200 mg/m², administered orally every 72 hours; 250 mg/m², administered orally every 72 hours; 275 mg/m², administered orally every 72 hours; 300 mg/m², administered orally every 72 hours; 350 mg/m², administered orally every 72 hours; 400 mg/m², administered orally every 72 hours; 425 mg/m², administered orally every 72 hours; 450 mg/m², administered orally every 72 hours; 500 mg/m², administered orally every 72 hours; 550 mg/m², administered orally every 72 hours; 600 mg/m², administered orally every 72 hours; 650 mg/m², administered orally every 72 hours; 700 mg/m², administered orally every 72 hours; 750 mg/m², administered orally every 72 hours; 800 mg/m², administered orally every 72 hours; 100 mg/m², administered orally every 84 hours; 125 mg/m², administered orally every 84 hours; 150 mg/m², administered orally every 84 hours; 200 mg/m², administered orally every 84 hours; 250 mg/m², administered orally every 84 hours; 275 mg/m², administered orally every 84 hours; 300 mg/m², administered orally every 84 hours; 350 mg/m², administered orally every 84 hours; 400 mg/m², administered orally every 84 hours; 425 mg/m², administered orally every 84 hours; 450 mg/m², administered orally every 84 hours; 500 mg/m², administered orally every 84 hours; 550 mg/m², administered orally every 84 hours; 600 mg/m², administered orally every 84 hours; 650 mg/m², administered orally every 84 hours; 700 mg/m², administered orally every 84 hours; 750 mg/m², administered orally every 84 hours; 800 mg/m², administered orally every 84 hours; 100 mg/m², administered orally every 96 hours; 125 mg/m², administered orally every 96 hours; 150 mg/m², administered orally every 96 hours; 200 mg/m², administered orally every 96 hours; 250 mg/m², administered orally every 96 hours; 275 mg/m², administered orally every 96 hours; 300 mg/m², administered orally every 96 hours; 350 mg/m², administered orally every 96 hours; 400 mg/m², administered orally every 96 hours; 425 mg/m², administered orally every 96 hours; 450 mg/m², administered orally every 96 hours; 500 mg/m², administered orally every 96 hours; 550 mg/m², administered orally every 96 hours; 600 mg/m², administered orally every 96 hours; 650 mg/m², administered orally every 96 hours; 700 mg/m², administered orally every 96 hours; 750 mg/m², administered orally every 96 hours; or 800 mg/m², administered orally every 96 hours.

Minimum and maximum threshold levels of a metabolite depend on a variety of factors, such as the type of subject, metabolite, therapeutic agent, and type of sample. Minimum and maximum threshold levels may be expressed in absolute terms, e.g., in units of concentration, or in relative terms, e.g., in ratios relative to a baseline or reference value. For example, the minimum threshold (below which a patient may receive a dose increase or additional dose) could also be calculated in terms of increase from a pre-treatment DHO level or baseline level.

Minimum threshold levels of DHO or orotate in a human plasma sample may be about 0 ng/ml, about 10 ng/mL, about 20 ng/mL, about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1250 ng/ml, about 1500 ng/ml, about 1750 ng/ml, about 2000 ng/ml, about 2500 ng/ml, about 3000 ng/ml, about 3500 ng/ml, about 4000 ng/ml, about 4500 ng/ml, about 5000 ng/ml, about 6000 ng/ml, about 8000 ng/ml, about 10,000 ng/ml, about 12,000 ng/ml, about 15,000 ng/ml, about 20,000 ng/ml, about 25,000 ng/ml, about 30,000 ng/ml, about 40,000 ng/ml, about 50,000 ng/ml, about 75,000 ng/ml, about 100,000 ng/ml, about 150,000 ng/ml, about 200,000 ng/ml, about 300,000 ng/ml, or about 400,000 ng/ml. The minimum threshold may include any value that falls between the values recited above. Thus, the minimum threshold may include any value between 0 ng/ml and 400.00 ng/ml.

Maximum threshold levels of DHO or orotate in a human plasma sample may be about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1250 ng/ml, about 1500 ng/ml, about 1750 ng/ml, about 2000 ng/ml, about 2500 ng/ml, about 3000 ng/ml, about 3500 ng/ml, about 4000 ng/ml, about 4500 ng/ml, about 5000 ng/ml, about 6000 ng/ml, about 8000 ng/ml, about 10,000 ng/ml, about 12,000 ng/ml, about 15,000 ng/ml, about 20,000 ng/ml, about 25,000 ng/ml, about 30,000 ng/ml, about 40,000 ng/ml, about 50,000 ng/ml, about 75,000 ng/ml, about 100,000 ng/ml, about 150,000 ng/ml, about 200,000 ng/ml, about 300,000 ng/ml, about 400,000 ng/ml, or about 500,000 ng/ml. The maximum threshold may include any value that falls between the values recited above. Thus, the maximum threshold may include any value between 50 ng/ml and 500.00 ng/ml.

The minimum threshold of DHO or orotate may be about 1.5 times the baseline level, about 2 times the baseline level, about 2.5 times the baseline level, about 3 times the baseline level, about 4 times the baseline level, about 5 times the baseline level, about 10 times the baseline level, about 20 times the baseline level, about 50 times the baseline level, about 100 times the baseline level, about 200 times the baseline level, about 500 times the baseline level, about 1000 times the baseline level, about 2000 times the baseline level, or about 5000 times the baseline level. The minimum threshold may include any ratio that falls between those recited above. Thus, the minimum threshold may be any ratio between 1.5 times the baseline level and 5000 times the baseline level.

The maximum threshold of DHO or orotate may be about 2 times the baseline level, about 2.5 times the baseline level, about 3 times the baseline level, about 4 times the baseline level, about 5 times the baseline level, about 10 times the baseline level, about 20 times the baseline level, about 50 times the baseline level, about 100 times the baseline level, about 200 times the baseline level, about 500 times the baseline level, about 1000 times the baseline level, about 2000 times the baseline level, about 5000 times the baseline level, or about 10,000 times the baseline level. The maximum threshold may include any ratio that falls between those recited above. Thus, the maximum threshold may be any ratio between 2 times the baseline level and 10,000 times the baseline level.

The agent may be any agent that alters activity of a metabolic pathway. Preferably, the agent is an inhibitor of an enzyme in a metabolic pathway. Several inhibitors of enzymes in the pyrimidine synthesis pathway are known in the art. Inhibitors of DHODH include brequinar, leflunomide, and teriflunomide. Brequinar, which has the systematic name 6-fluoro-2-(2′-fluoro-1,1′ biphenyl-4-yl)-3-methyl-4-quinoline carboxylic acid, has the following structure:

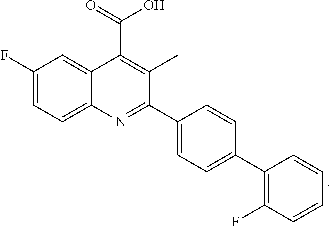 [SMILES: Cc1c(-c2ccc(-c3ccccc3F)cc2)nc2ccc(F)cc2c1C(=O)O]

Brequinar and related compounds are described in, for example, U.S. Pat. Nos. 4,680,299 and 5,523,408, the contents of which are incorporated herein by reference. The use of brequinar to treat leukemia is described in, for example, U.S. Pat. No. 5,032,597 and WO 2017/037022, the contents of which are incorporated herein by reference. Leflunomide, N-(4′-trifluoromethylphenyl)-5-methylisoxazole-4-carboxamide (I), is described in, for example, U.S. Pat. No. 4,284,786, the contents of which are incorporated herein by reference. Teriflunomide, 2-cyano-3-hydroxy-N-[4-(trifluoromethyl)phenyl]-2-butenamide, is described in, for example, U.S. Pat. No. 5,679,709, the contents of which are incorporated herein by reference. OMP decarboxylase inhibitors include pyrazofurin. Pyrazofurin, 5-[(2S,3R,4S,5R)-3,4-dihydroxy-5-(hydroxymethyl)oxolan-2-yl]-4-hydroxy-1H-pyrazole-3-carboxamide, has the following structure:

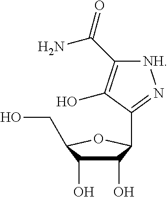

Pyrazofurin and related compounds are described in, for example, U.S. Pat. Nos. 3,674,774 and 3,802,999, the contents of which are incorporated herein by reference. ATCase inhibitors include N-(phosphonacetyl)-L-aspartate (PALA). PALA is described in, for example, Swyryd et al, N-(Phosphonacetyl)-L-Aspartate, a Potent Transition State Analog Inhibitor of Aspartate Transcarbamylase, Blocks Proliferation of Mammalian Cells in Culture, J. Biol. Chem. Vol. 249, No. 21, Issue of November 10, pp. 6945-6950, 1974.

Dosing of the agent may account for the formulation of the agent. For example, therapeutic agents, such as brequinar, pyrazofurin, leflunomide, teriflunomide, and PALA, may be provided as prodrugs, analogs, derivatives, or salts. Any of the aforementioned chemical forms may be provided in a pharmaceutically acceptable formulation, such as a micellar formulation.

Dosage of the agent also depends on factors such as the type of subject and route of administration. The dosage may fall within a range for a given type of subject and route of administration, or the dosage may adjusted by a specified amount for a given type of subject and route of administration. For example, dosage of brequinar for oral or intravenous administration to a subject, such as human or mouse, may be about 1 mg/kg, about 2 mg/kg, about 5 mg/kg, about 7.5 mg/kg, about 10 mg/kg, about 15 mg/kg, about 20 mg/kg, about 25 mg/kg, about 50 mg/kg, about 75 mg/kg, or about 100 mg/kg. Dosage of brequinar for oral or intravenous administration to a subject, such as human or mouse, may be adjusted by about 1 mg/kg, about 2 mg/kg, about 5 mg/kg, about 7.5 mg/kg, about 10 mg/kg, about 15 mg/kg, about 20 mg/kg, about 25 mg/kg, or about 50 mg/kg. Dosage of brequinar for oral or intravenous administration to an animal subject, such as a human or mouse, may be about 50 mg/m², about 100 mg/m², about 200 mg/m², about 300 mg/m², about 350 mg/m², about 400 mg/m², about 500 mg/m², about 600 mg/m², about 700 mg/m², about 750 mg/m², about 800 mg/m², or about 1000 mg/m². Dosage of brequinar for oral or intravenous administration to an animal subject, such as a human or mouse, may be adjusted by about 50 mg/m², about 100 mg/m², about 200 mg/m², about 300 mg/m², about 350 mg/m², or about 400 mg/m².

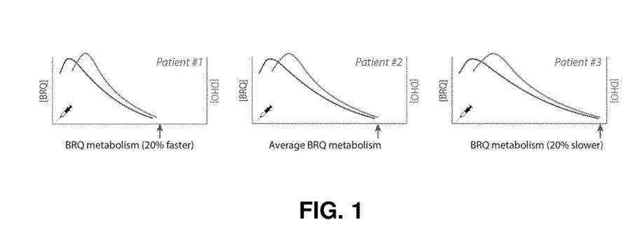

FIG. 1 is a series of graphs showing levels of brequinar and DHO in three patients that have received a single dose of brequinar according to the same dosing regimen. The graph on the left is from patient #1, the graph in center is from patient #2, and the graph on the right is from patient #3. Levels of brequinar are shown in dark green, and levels of DHO are shown in red. Metabolism of brequinar is faster than average in patient #1, average in patient #2, and slower than average in patient #3. Inhibition of DHODH leads to accumulation of DHO, a substrate of DHODH. However, analysis of brequinar levels alone provides an incomplete picture of the efficacy of brequinar. Because analysis of DHO levels gives a more accurate representation of target engagement, DHO is a superior biomarker.

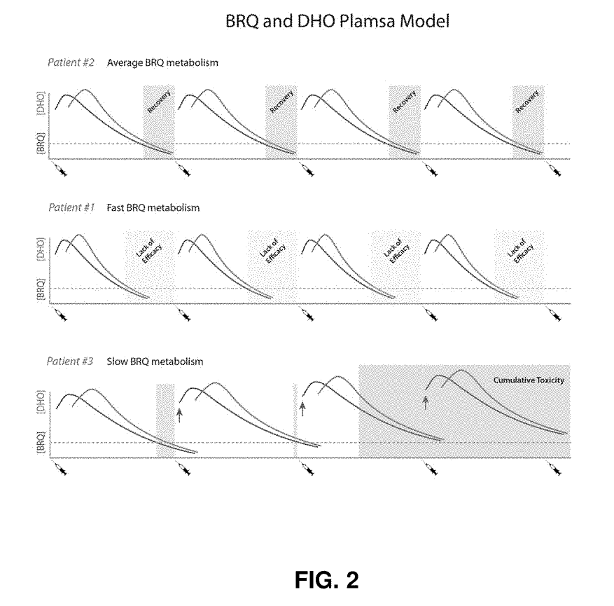

FIG. 2 is a series of graphs showing levels of brequinar and DHO in three patients that have received a multiple doses of brequinar according to the same dosing regimen. The graph on the top is from patient #2, the graph in center is from patient #1, and the graph on the bottom is from patient #3. Levels of brequinar are shown in dark green, levels of DHO are shown in red, and the dashed line represents a threshold level above which brequinar provides sufficient inhibition of DHODH. In patient #2, i.e., a patient with an average rate of brequinar metabolism, the dosing regimen provides periods of sustained inhibition of DHODH interspersed with short recovery periods. This dosing regimen is optimal for patient #2 because the prolonged inhibition of DHODH kills leukemia cells that are sensitive to uridine starvation, while the recovery period allows an adequate supply of pyrimidines to support survival of normal cells. In patient #1, however, the duration of DHODH inhibition is not sufficient to kill leukemia cells, so this dosing regimen does not provide a therapeutic benefit. Conversely, in patient #3, the second and subsequent doses of brequinar are provided too shortly after DHODH activity is restored following the previous dose, and the pyrimidine pool is not adequately restored to support survival of normal cells. Consequently, this dosing regimen is toxic to patient #3.

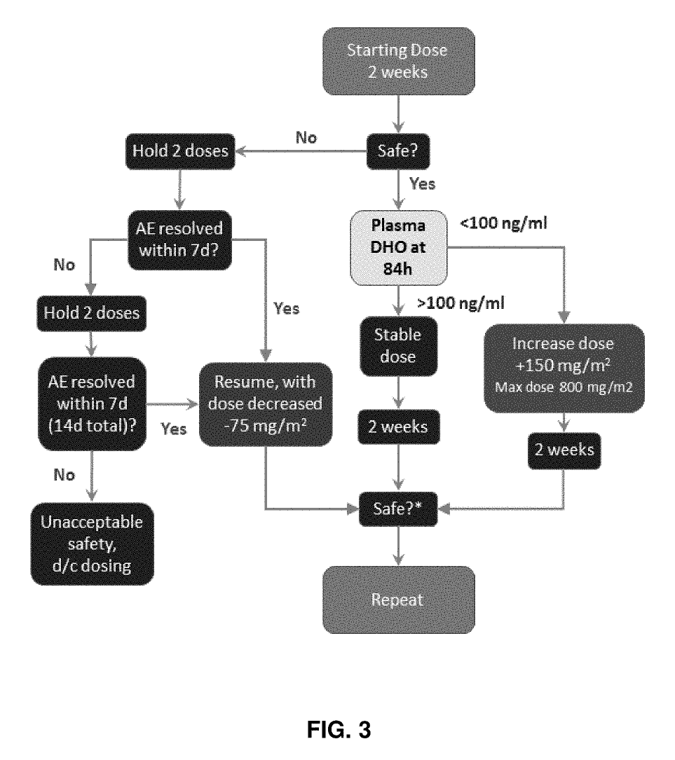

FIG. 3 is a flow chart illustrating an example of determining a dose a of DHODH inhibitor for a patient according to an embodiment of the invention. A pre-treatment DHO level is measured to determine the DHO baseline for the patient. The patient is given a starting dose for 2 weeks and examined for the presence of adverse events (AE). If adverse events occur, subsequent doses are withheld to see whether the adverse events resolve within 7 days. If adverse events resolve, dosage is decreased by 75 mg/m² and dosing is resumed. If no adverse events occur, DHO levels are analyzed at 84 hours post-administration. If DHO levels are below 100 ng/mL or two times the baseline, dosage of brequinar is increased by 150 mg/m² but not to exceed a maximum dosage of 800 mg/m². If DHO levels are above 100 ng/mL, the dosing is maintained for 2 weeks. The process can be repeated to optimize the dosing to achieve sustained elevation of DHO levels above the threshold level without adverse events.

The methods are useful for providing guidance on dosing of therapeutic agents for individuals. Therefore, the methods may be performed by any party that wishes to provide such guidance. For example and without limitation, the methods may be performed by a clinical laboratory; a physician or other medical professional; a supplier or manufacturer of a therapeutic agent; an organization that provides analytical services to a physician, clinic, hospital, or other medical service provider; or a healthcare consultant.

Disorders that can be Treated by Altering Activity of a Metabolic Pathway

The methods of the invention are useful for determining the dosage of drugs that affect that alter the activity of a metabolic pathway to treat or prevent a disorder. Preferably, the drug inhibits an enzyme in the metabolic pathway. In other embodiments, the drug inhibits an enzyme in a related metabolic pathway, such as a pathway that regulates, compensates for, or antagonizes the pathway in which the target enzyme functions. Thus, the disorder may be any disease, disorder, or condition for which enzyme inhibition provides a therapeutic benefit.

For example and without limitation, one disorder that can be treated by methods of the invention is acute myeloid leukemia (AML). In AML, myeloblasts arrested in an early stage of differentiation proliferate in an uncontrolled manner and interfere with the development of other blood cells in the bone marrow. Inhibitors of dihydroorotate dehydrogenase (DHODH), an enzyme involved in pyrimidine synthesis, cause differentiation of myeloblasts and prevent their leukemia-initiating activity. The role of DHODH in AML is described in Sykes et al., Inhibition of Dihydroorotate Dehydrogenase Overcomes Differentiation Blockade in Acute Myeloid Leukemia, Cell 167, 171-186, Sep. 22, 2016; dx.doi.org/10. 1016/j.cell.2016.08.057, the contents of which are incorporate herein by reference.

The use of DHODH inhibitors to treat AML requires a precise dosing regimen. Care must be taken to avoid excessive inhibition of DHODH. DHODH is an essential enzyme, and homozygous recessive mutations in DHODH cause Miller syndrome, a disorder characterized by multi-organ dysfunction. In a mouse model of AML, daily administration of high doses of the DHODH inhibitor brequinar lead to weight loss, anemia, and thrombocytopenia. At the same time, sustained exposure to brequinar is necessary to inhibit DHODH for sufficient periods to produce a therapeutic effect in the mouse AML model. Without wishing to be bound by theory, one hypothesis for the narrow therapeutic window of brequinar in treating AML in both the mouse model and in humans is that malignant cells display an increased sensitivity to DHODH inhibition. In particular, normal cells may be able to tolerate periods of nucleotide starvation that kill cancer cells due to the elevated metabolic needs of the latter.

The narrow therapeutic window of DHODH inhibition likely applies to other disorders as well. For example, brequinar was evaluated for treatment of solid tumor malignancies and found to be ineffective when administered over a 5-day period followed by a 3-week gap or once per week for three weeks followed by a 1-week gap. See Arteaga, C. L. et al. (1989) Phase I clinical and pharmacokinetic trial of Brequinar sodium (DuP 785; NSC 368390) Cancer Res. 49, 4648-4653; Burris, H. A., et al. (1998) Pharmacokinetic and phase I studies of brequinar (DUP 785; NSC 368390) in combination with cisplatin in patients with advanced malignancies, Invest. New Drugs 16, 19-27; Noe, D. A., et al. (1990) Phase I and pharmacokinetic study of brequinar sodium (NSC 368390), Cancer Res. 50, 4595-4599; Schwartsmann, G. et al. (1990) Phase I study of Brequinar sodium (NSC 368390) in patients with solid malignancies, Cancer Chemother. Pharmacol. 25, 345-351, the contents of each of which are incorporated herein by reference. However, brequinar may be effective for treatment of other cancers if the drug is administered in a manner that provides sustained DHODH inhibition.

It is understood that the aforementioned examples are provided for illustrative purposes only and that the methods of the invention can be used for treatment of any disorder or disease in which the measured level of a metabolite can be used to assess target engagement. The disorder may be one in which inhibiting an enzyme in a metabolic pathway is of therapeutic benefit. The disorder may be cancer. The cancer may include a solid tumor or hematological tumor. The cancer may be acute lymphoblastic leukemia (ALL), adult T cell leukemia/lymphoma (ATLL), bladder cancer, breast cancer, such as triple negative breast cancer (TNBC), glioma, head and neck cancer, leukemia, such as AML, lung cancer, such as small cell lung cancer and non-small cell lung cancer, lymphoma, multiple myeloma, neuroblastoma, osteosarcoma, ovarian cancer, prostate cancer, or renal cell cancer. The disorder may have a genetic mutation such as MYC amplification or PTEN loss that leads to increased dependence on the metabolic pathway, such as increased “addiction” to glutamine. The disorder may be an inflammatory or autoimmune disorder, such as arthritis, hepatitis, chronic obstructive pulmonary disease, multiple sclerosis, or tendonitis. The disorder may be a psychiatric disorder, such as anxiety, stress, obsessive-compulsive disorder, depression, panic disorder, psychosis, addiction, alcoholism, attention deficit hyperactivity, agoraphobia, schizophrenia, or social phobia. The disorder may be a neurological or pain disorder, such as epilepsy, stroke, insomnia, diskinesia, peripheral neuropathic pain, chronic nociceptive pain, phantom pain, deafferentation pain, inflammatory pain, joint pain, wound pain, post-surgical pain, or recurrent headache pain, appetite disorders, or motor activity disorders. The disorder may be a neurodegenerative disorder, such as Alzheimer's disease, Parkinson's disease, or Huntington's disease.

The disorder may include a class or subset of patients having a disease, disorder, or condition. For example, AML cases are classified based on cytological, genetic, and other criteria, and AML treatment strategies vary depending on classification. One AML classification system is provided by the World Health Organization (WHO). The WHO classification system includes subtypes of AML provided in Table 1 and is described in Falini B, et al. (October 2010) “New classification of acute myeloid leukemia and precursor-related neoplasms: changes and unsolved issues” Discov Med. 10 (53): 281-92, PMID 21034669, the contents of which are incorporated herein by reference.

TABLE 1

| Name | Description |
|---|---|
| Acute myeloid | Includes: |
| leukemia with | AML with translocations between chromosome 8 and 21 - |
| recurrent | [t(8; 21)(q22; q22);] RUNX1/RUNX1T1; (ICD-O 9896/3); |
| genetic | AML with inversions in chromosome 16 - [inv(16)(p13.1q22)] or internal |
| abnormalities | translocations in it - [t(16; 16)(p13.1; q22);] CBFB/MYH11; (ICD-O |
| | 9871/3); |
| | Acute promyelocytic leukemia with translocations between chromosome |
| | 15 and 17 - [t(15; 17)(q22; q12);] RARA/PML; (ICD-O 9866/3); |
| | AML with translocations between chromosome 9 and 11 - |
| | [t(9; 11)(p22; q23);] MLLT3/MLL; |
| | AML with translocations between chromosome 6 and 9 - |
| | [t(6; 9)(p23; q34);] DEK/NUP214; |
| | AML with inversions in chromosome 3 - [inv(3)(q21q26.2)] or internal |
| | translocations in it - [t(3; 3)(q21; q26.2);] RPN1/EVI1; |
| | Megakaryoblastic AML with translocations between chromosome |
| | 1 and 22 - [t(1; 22)(p13; q13);] RBM15/MKL1; |
| | AML with mutated NPM1 |
| | AML with mutated CEBPA |
| AML with | Includes people who have had a prior documented myelodysplastic |
| myelodysplasia- | syndrome (MDS) or myeloproliferative disease (MPD) that then has |
| related changes | transformed into AML, or who have cytogenetic abnormalities characteristic |
| | for this type of AML (with previous history of MDS or MPD that has gone |
| | unnoticed in the past, but the cytogenetics is still suggestive of MDS/MPD |
| | history). This category of AML occurs most often in elderly people and often |
| | has a worse prognosis. Includes: |
| | AML with complex karyotype |
| | Unbalanced abnormalities |
| | AML with deletions of chromosome 7 - [del(7q);] |
| | AML with deletions of chromosome 5 - [del(5q);] |
| | AML with unbalanced chromosomal aberrations in chromosome 17 - |
| | [i(17q)/t(17p);] |
| | AML with deletions of chromosome 13 - [del(13q);] |
| | AML with deletions of chromosome 11 - [del(11q);] |
| | AML with unbalanced chromosomal aberrations in chromosome 12 - |
| | [del(12p)/t(12p);] |
| | AML with deletions of chromosome 9 - [del(9q);] |
| | AML with aberrations in chromosome X - [idic(X)(q13);] |
| | Balanced abnormalities |
| | AML with translocations between chromosome 11 and 16 - |
| | [t(11; 16)(q23; q13.3);], unrelated to previous chemotherapy or |
| | ionizing radiation |
| | AML with translocations between chromosome 3 and 21 - |
| | [t(3; 21)(q26.2; q22.1);], unrelated to previous chemotherapy or |
| | ionizing radiation |
| | AML with translocations between chromosome 1 and 3 - |
| | [t(1; 3)(p36.3; q21.1);] |
| | AML with translocations between chromosome 2 and 11 - |
| | [t(2; 11)(p21; q23);], unrelated to previous chemotherapy or ionizing |
| | radiation |
| | AML with translocations between chromosome 5 and 12 - |
| | [t(5; 12)(q33; p12);] |
| | AML with translocations between chromosome 5 and 7 - |
| | [t(5; 7)(q33; q11.2);] |
| | AML with translocations between chromosome 5 and 17 - |
| | [t(5; 17)(q33; p13);] |
| | AML with translocations between chromosome 5 and 10 - |
| | [t(5; 10)(q33; q21);] |
| | AML with translocations between chromosome 3 and 5 - |
| | [t(3; 5)(q25; q34);] |
| Therapy-related | Includes people who have had prior chemotherapy and/or radiation and |
| myeloid | subsequently develop AML or MDS. These leukemias may be characterized |
| neoplasms | by specific chromosomal abnormalities, and often carry a worse prognosis. |
| Myeloid | Includes myeloid sarcoma. |
| sarcoma | |
| Myeloid | Includes so-called “transient abnormal myelopoiesis” and “Myeloid leukemia |
| proliferations | associated with Down syndrome” |
| related to Down | |
| syndrome | |
| Blastic | Includes so-called “blastic plasmacytoid dendritic cell neoplasm” |
| plasmacytoid | |
| dendritic cell | |
| neoplasm | |
| AML not | Includes subtypes of AML that do not fall into the above categories |
| otherwise | AML with minimal differentiation |
| categorized | AML without maturation |
| | AML with maturation |
| | Acute myelomonocytic leukemia |
| | Acute monoblastic and monocytic leukemia |
| | Acute erythroid leukemia |
| | Acute megakaryoblastic leukemia |
| | Acute basophilic leukemia |
| | Acute panmyelosis with myelofibrosis |

An alternative classification scheme for AML is the French-American-British (FAB) classification system. The FAB classification system includes the subtypes of AML provided in Table 2 and is described in Bennett J M, et al. (August 1976). “Proposals for the classification of the acute leukaemias. French-American-British (FAB) co-operative group” Br. J. Haematol. 33 (4): 451-8, doi:10.1111/j.1365-2141.1976.tb03563.x. PMID 188440; and Bennett J M, et al. (June 1989) “Proposals for the classification of chronic (mature) B and T lymphoid leukaemias. French-American-British (FAB) Cooperative Group” J. Clin. Pathol. 42 (6): 567-84, doi:10.1136/jcp.42.6.567, PMC 1141984, PMID 2738163, the contents of each of which are incorporated herein by reference.

TABLE 2

| Type | Name | Cytogenetics |
|---|---|---|
| M0 | acute myeloblastic leukemia, minimally | |
| | differentiated | |
| M1 | acute myeloblastic leukemia, without | |
| | maturation | |
| M2 | acute myeloblastic leukemia, with | t(8; 21)(q22; q22), |
| | granulocytic maturation | t(6; 9) |
| M3 | promyelocytic, or acute promyelocytic | t(15; 17) |
| | leukemia (APL) | |
| M4 | acute myelomonocytic leukemia | inv(16)(p13q22), |
| | | del(16q) |
| M4eo | myelomonocytic together with bone | inv(16), t(16; 16) |
| | marrow eosinophilia | |
| M5 | acute monoblastic leukemia (M5a) or acute | del (11q), t(9; 11), |
| | monocytic leukemia (M5b) | t(11; 19) |
| M6 | acute erythroid leukemias, including | |
| | erythroleukemia (M6a) and very rare pure | |
| | erythroid leukemia (M6b) | |
| M7 | acute megakaryoblastic leukemia | t(1; 22) |

The disorder may include a sub-population of patients. For example, the patients may be pediatric, newborn, neonates, infants, children, adolescent, pre-teens, teenagers, adults, or elderly. The patients may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

### Providing Doses of a Therapeutic Agent

Methods of the invention may include providing a therapeutic agent to a subject according to a dosing regimen or dosage determined as described above. Providing the agent to the subject may include administering it to the subject. A dose may be administered as a single unit or in multiple units. The agent may be administered by any suitable means. For example and without limitation, the agent may be administered orally, intravenously, enterally, parenterally, dermally, buccally, topically, transdermally, by injection, intravenously, subcutaneously, nasally, pulmonarily, or with or on an implantable medical device (e.g., stent or drug-eluting stent or balloon equivalents).

In some embodiments, the methods include assessing a metabolite level in a sample from a subject, and determining whether that level is within a threshold range (e.g., above a minimal threshold and/or below a potential toxicity threshold) that warrants dosing, and/or that warrants dosing at a particular level or in a particular amount.

The methods may include administering at least one dose of the agent to a subject whose plasma metabolite level has been determined and is below a pre-determined threshold (e.g., a pre-determined potential toxicity threshold and/or a pre-determined potential efficacy threshold). In some embodiments, the predetermined threshold reflects percent inhibition of a target enzyme in the subject relative to a baseline determined for the subject. In some embodiments, the baseline is determined by an assay.

For example, in some embodiments, in order to maintain inhibition of the target enzyme at an effective threshold, multiple doses of the agent may be administered. In some embodiments, dosing of the agent can occur at different times and in different amounts. The present disclosure encompasses those methods that can maintain inhibition of the target enzyme at a consistent level at or above the efficacy threshold throughout the course of treatment. In some embodiments, the amount of inhibition of the target enzyme is measured by the amount of metabolite in the plasma of a subject.

In some embodiments, more than one dose of the agent is administered to the subject. In some embodiments, the method further comprises a step of re-determining the subject's plasma metabolite level after administration of the at least one dose. In some embodiments, the subject's plasma metabolite level is re-determined after each dose. In some embodiments, the method further comprises administering at least one further dose of the agent after the subject's plasma metabolite level has been determined again (e.g., after administering a first or previous dose), and is below the pre-determined threshold. If the subject's plasma metabolite level is determined to be above a pre-determined threshold, dosing can be discontinued. In some embodiments, therefore, no further dose of the agent is administered until the subject's plasma metabolite level has been determined to again be below a pre-determined threshold.

The methods may include administering an agent to a subject at a dosage level at or near a cell-lethal level. Such dosage can be supplemented with a later dose at a reduced level, or by discontinuing of dosing. For example, in some embodiments, the present disclosure provides a method of administering a dihydroorotate dehydrogenase inhibitor to a subject in need thereof, the method comprising: administering a plurality of doses of an agent, according to a regimen characterized by at least first and second phases, wherein the first phase involves administration of at least one bolus dose of an agent at a cell-lethal level; and the second phase involves either: administration of at least one dose that is lower than the bolus dose; or absence of administration of an agent.

In some embodiments, an agent is not administered during a second phase. In some embodiments, a second phase involves administration of uridine rescue therapy. In some embodiments, a bolus dose is or comprises a cell lethal dose. In some embodiments, a cell lethal dose is an amount of an agent that is sufficient to cause apoptosis in normal (e.g., non-cancerous) cells in addition to target cells (e.g., cancer cells).

In some embodiments, the first phase and the second phase each comprise administering an agent. In some embodiments, the first phase and the second phase are at different times. In some embodiments, the first phase and the second phase are on different days. In some embodiments, the first phase lasts for a period of time that is less than four days. In some embodiments, the first phase comprises administering an agent, followed by a period of time in which no agent is administered. In some embodiments, the period of time in which no agent is administered is 3 to 7 days after the dose during the first phase. In some embodiments, the first phase comprises administering more than one dose.

In some embodiments, an agent is administered during a second phase. In some embodiments, an agent is administered sub-cell-lethal levels during the second phase. In some embodiments, the first phase is repeated after the second phase. In some embodiments, both the first and second phases are repeated.

In some embodiments, the present disclosure provides a method of administering an agent to a subject in need thereof, according to a multi-phase protocol comprising: a first phase in which at least one dose of the agent is administered to the subject; and a second phase in which at least one dose of the agent is administered to the subject, wherein one or more doses administered in the second phase differs in amount and/or timing relative to other doses in its phase as compared with the dose(s) administered in the first phase.

In some embodiments, a metabolite level is determined in a sample from the subject between the first and second phases. In some embodiments, the sample is a plasma sample. In some embodiments, the timing or amount of at least one dose administered after the metabolite level is determined or differs from that of at least one dose administered before the metabolite level was determined.

In some embodiments, the amount of agent that is administered to the patient is adjusted in view of the metabolite level in the subject's plasma. For example, in some embodiments, a first dose is administered in the first phase. In some embodiments, metabolite level is determined at a period of time after administration of the first dose.

In some embodiments, if the metabolite level is below a pre-determined level, the amount of agent administered in a second or subsequent dose is increased and/or the interval between doses is reduced. For example, in some such embodiments, the amount of agent administered may be increased, for example, by 100 mg/m². In some embodiments, the amount of agent administered in a second or subsequent dose is increased by 150 mg/m². In some embodiments, the amount of agent administered in a second or subsequent dose is increased by 200 mg/m². In some embodiments, the amount of agent administered may be increased by an adjustment amount determined based on change in metabolite levels observed between prior doses of different amounts administered to the subject.

In some embodiments, if the metabolite level is above a pre-determined level, the amount of agent administered in a second or subsequent dose is the same as the amount administered in the first or previous dose and/or the interval between doses is the same.

In some embodiments, if the metabolite level is above a pre-determined level, the amount of agent in a second or subsequent dose is decreased and/or the interval between doses is increased. For example, in some such embodiments, the amount of agent administered may be decreased, for example, by 50 mg/m². In some embodiments, if the metabolite level is above a pre-determined level, the amount of agent in a second or subsequent dose is decreased by 75 mg/m². In some embodiments, if the metabolite level is above a pre-determined level, the amount of agent in a second or subsequent dose is decreased by 100 mg/m². In some embodiments, the amount of agent administered may be decreased by an adjustment amount determined based on change in metabolite levels observed between prior doses of different amounts administered to the subject.

In some embodiments, the present disclosure provides a method of administering a later dose of an agent to a patient who has previously received an earlier dose of the agent, wherein the patient has had a level of metabolite assessed subsequent to administration of the earlier dose, and wherein the later dose is different than the earlier dose. The later dose may be different from the earlier dose in amount of agent included in the dose, time interval relative to an immediately prior or immediately subsequent dose, or combinations thereof. The amount of agent in the later dose may be less than that in the earlier dose.

The method may include administering multiple dose of the agent, separated from one another by a time period that is longer than 2 days and shorter than 8 days For example, the time period may be about 3 days.

In some embodiments, the metabolite level is determined in a sample from the subject before each dose is administered, and dosing is delayed or skipped if the determined metabolite level is above a pre-determined threshold. For example, the metabolite level may be determined about 12 hours, about 24 hours, about 36 hours, about 48 hours, about 60 hours, about 72 hours, about 84 hours, or about 96 hours after administration of an agent

The method may include administering the agent according to a regimen approved in a trial in which a level of metabolite was measured in a patients between doses of the agent The regimen may include multiple doses whose amount and timing were determined in the trial to maintain the metabolite level within a range determined to indicate a degree of target enzyme inhibition below a toxic threshold and above a minimum threshold. The regimen may include determining the metabolite level in the subject after administration of one or more doses of the agent.

In some embodiments, the regimen includes a dosing cycle in which an established pattern of doses is administered over a first period of time. In some embodiments, the regimen comprises a plurality of the dosing cycles. In some embodiments, the regimen includes a rest period during which the agent is not administered between the cycles.

### Assessing Tumor Properties

The invention also provides methods for assessing the effects of therapeutic agents on tumors in vivo in real time. This information obtained from such in vivo analysis may be used to determine or make adjustments to dosing regimens.

One modality for assessing the effect of an agent on a tumor is to monitor within the tumor the flux of a metabolite through a pathway whose activity is altered by the agent, such as the pathways and agents described above. Activity of metabolic pathways in vivo can be analyzed in real-time by hyperpolarization magnetic resonance imaging, as described in, for example, Miloushev, V Z et al., Hyperpolarization MRI: Preclinical Models and Potential Applications in Neuroradiology, Top Magn Reson Imaging 2016 February; 25(1): 31-37, doi: 10.1097/RMR.0000000000000076, PMID: 26848559; and Di Gialleonardo, D, et al., The Potential of Metabolic Imaging, Semin Nucl Med. 2016 January; 46(1): 28-39, doi: 10.1053/j.semnuclmed.2015.09.004, PMID: 26687855; and Cho, et al., Noninvasive Interrogation of Cancer Metabolism with Hyperpolarized 13C MRI J Nucl Med 2017; 58:1201-1206, DOI: 10.2967/jnumed. 116.182170, the contents of each of which are incorporated herein by reference.

Briefly, the methods entail injection of an isotopically-labeled metabolite, which can be imaged by magnetic resonance, into a subject and tracking movement of the isotope through the body. The metabolite may be a carbon-containing molecule, such as an intermediate in the pyrimidine synthesis pathway, that is enriched for an isotope of carbon, such as 13C, or nitrogen, such as 15N. The therapeutic agent may be an agent that inhibits an enzyme in a pathway through which the metabolite passes. Analysis may include comparison of metabolism of the labeled metabolite when the subject has been provided the therapeutic agent with metabolism in an untreated subject, either the same subject or a different subject having similar characteristics. The methods are useful for analysis of tumors due to the increase flux through certain metabolic pathways, such as the pyrimidine synthesis pathway, in tumor cells. For example, a subject having a tumor with increased glutamine flux (determined by isotopically-labeled glutamine) may be given a DHODH inhibitor, e.g., brequinar, and isotopically-labeled DHO. If the level of DHODH inhibition is high, accumulation of the metabolite can be detected at the site of the tumor.

Another way to assess the effect of an agent on a tumor in vivo in real time is to analyze oxygenation of the tumor. Many solid tumors contain regions of poor oxygenation due to the inability of the vasculature to keep pace with the rapid growth of tumor cells. To continue to proliferating when the blood supply is inadequate, tumor cells often alter their metabolism to derive more energy from glucose metabolism and become less dependent on oxygen. Methods of measuring oxygenation levels of tissue that contains tumors is known in the art and described in, for example, Zhao, D., et al., Measuring changes in tumor oxygenation, Methods Enzymol. 2004; 386:378-418, doi.org/10.1016/S0076-6879(04)86018-X; and H Rundqvist and R S Johnson, Tumour oxygenation: implications for breast cancer prognosis, Intern Med 2013; 274: 105-112, doi: 10.1111/joim.12091, the contents of each of which are incorporated herein by reference. In some embodiments, tumor oxygenation may be measured by electron paramagnetic resonance imaging (EPR). EPR is known in the art and described in, for example, Abramovic Z., et al., (eds) 11th Mediterranean Conference on Medical and Biomedical Engineering and Computing 2007. IFMBE Proceedings, vol 16. Springer, Berlin, Heidelberg, doi.org/10.1007/978-3-540-73044-6_116, ISBN 978-3-540-73043-9; and Matsumoto, et al., Low-field paramagnetic resonance imaging of tumor oxygenation and glycolytic activity in mice, J. Clin. Invest. 118:1965-1973 (2008) doi:10.1172/JCI34928, the contents of each of which are incorporated herein by reference.

### A Device to Rapidly Assess Metabolite Levels

The invention also includes a device or assay to rapidly measure levels of a metabolite of interest, for e.g., DHO. Plasma from a patient is run on the assay with the objective to determine the level of metabolite in the plasma. In the described assay, set levels of the target enzyme are added with known activity. The assay quantifies the amount of metabolite present in plasma by colorimetric changes, a competitive assay, or other techniques known in the field. In one embodiment, the objective is to quantify the amount of DHO after a dose of brequinar. A patient plasma specimen is collected. The plasma is run on the assay containing set amount of DHODH. Patient DHO may compete with colored DHO in the assay and cause a change in color that can be read out as a measure of DHO level in the plasma. In another embodiment, substrate and DHODH could be lyophilized in a blood collection tube. Blood drawn into the tube could provide a visible change in color to determine if DHO is below, at or above a specified threshold. This would enable point of care monitoring of metabolite levels for rapid adjustments in dose as needed.

### Devices for Notification

The invention also includes devices for notifying a subject concerning a dosing regimen, such as a dosage of a therapeutic agent, timing for administration of a dose, timing for collection of a metabolite to determine dose adjustments, or any combination thereof, or an adjustment to a dosing regimen. The devices include a processor coupled to a memory unit. The memory unit drives the processor to receive data about a dose of a therapeutic agent, collect data from laboratory or point of care analysis of the metabolite tested, generate a notification about a dosing regimen or a change to the dosing regimen, and output the reminder to the subject.

The data received by the processor may contain any information related to a dose of an agent provided to a subject. For example, the data may include information about the agent, such as the name of the agent, a classification the agent, the dose or amount of the agent provided to the subject, the concentration, the formulation, and the like. The data may include the route of administration, such as oral or intravenous administration. The data may include the when the dose was administered to the subject, including the day, date, hour, minute, second, time zone, or any other temporal component. The data may include information concerning multiple doses of the agent that were administered to the subject. The data may include information concerning multiple agents that were administered to the subject. The data may include a metabolite level and whether a specified threshold has been reached.

The notification may include any type of reminder to the subject concerning the dosing regimen or adjustments thereto. For example, the notification may include a time for administration of the next dose of the agent, the dosage of the next dose of the agent, or a combination of the two. The notification may include adjustments to any of the aforementioned parameters. The notification may include information provided in absolute terms or relative terms. For example, the notification may include a time component that indicate that the next dose should be provided at a certain number of hours, e.g., 72 hours, following the previous dose, or it may indicate an objective time and/or date for administration of the next dose. The notification may indicate that the dosage should be adjusted by a defined amount, e.g., increased by 75 ng/mL, by a relative amount, e.g., increased by 50%. The dosing regimen or adjustment to the dosing regimen is based on a measured level of a metabolite in a sample obtained from the subject, as described above. The notification may also recommend the time for an additional blood collection for metabolite analysis based on a trend analysis of historic drug and metabolite levels, a change in disease, or new evidence for an alternative blood sampling schedule.

The device may provide the notification in any manner that can be perceived by the subject. For example, output of the notification may include an audible signal, a visual signal, a tactile signal, a vibration, or any combination thereof.

The device may output the notification to a component of the device, such as a display, or it may output the notification to a remote device. The device may output the notification to a third party, such as health care professional, e.g., a physician, nurse, or other practitioner.

The memory unit may enable the processor to perform additional processes. For example, the processor may determine a dosing regimen or an adjustment to a dosing regimen, as described above.

The processor may use information stored in the memory unit to determine whether the subject has developed or is developing resistance to a therapeutic agent. Resistance of a subject to a therapeutic agent can become manifest when the interval between time points of dose administration to achieve the same effect, e.g., level of metabolite, become smaller over the course of therapy, i.e., when the subject requires more frequent doses. Resistance of a subject to a therapeutic agent can become manifest when higher dosages are required to achieve the same effect, e.g., level of metabolite, over the course of therapy. Thus, the processor may determine that intervals between time points for administration of the agent have changed, e.g., grown smaller or larger, over the course of therapy, that dosages have changed, e.g., increased or decreased, over the course of therapy, or a combination of the two.

The processor may output a recommended adjustment in the dosing regimen to the subject. The recommended adjustment may include administration of a second or additional therapeutic agent.

The device may be, or be a part of, a portable or wearable electronic device, such as a phone, watch, belt, armband, legband, article of clothing, handheld device, or the like.

### Synthetic Lethality

Methods of the invention include determining a dosing regimen that includes providing an agent that alters activity of a metabolic pathway in a tumor that is specifically dependent on that metabolic pathway. For example, tumor cells bearing a mutation that affects the activity of a first pathway may rely more heavily on the activity of a second pathway that compensates for or counteracts the altered activity of the first pathway. A change in the activity of the second pathway that may therefore be deadly to tumor cells but not to normal cells, a phenomenon called synthetic lethality. Examples of tumors with altered pathways for which a DHODH inhibitor, such as brequinar, may be synthetically lethal include tumors that have phosphatase and tensin homolog (PTEN) low, Myc protein family member amplification, a Notch protein family member mutations, and activating mutations of Ras protein family members.

### Combination Therapies for Autoimmune Toxicity

Methods of the invention include determining a dosing regimen that includes providing an agent that alters activity of a metabolic pathway, as described above, in combination with one or more other therapeutic agents. The methods may also include providing both therapeutic agents in such combination dosing regimens.

Combination therapies are useful, for example, for treating autoimmune toxicity and cytokine-associated toxicity. Autoimmune toxicity may result from an antigen-specific attack on host tissues when the targeted tumor associated antigen is expressed on nonmalignant tissue. It may result due to increased immune activation due to immunoncology (IO) therapy. It may preferentially affect patients with pre-existing autoimmune disease such as rheumatoid arthritis, inflammatory bowel disease, and psoriasis.

Cytokine Release Syndrome (CRS)

Cytokine associated toxicity, also referred to as cytokine release syndrome (CRS) or cytokine storm, is a non-antigen specific toxicity that occurs as a result of high level immune activation. The degree of immune activation necessary to obtain clinical benefit using IO typically exceeds the level of immune activation that occurs during natural immune activation. As IO therapies have increased in potency and efficacy, CRS is increasingly recognized as a problem requiring a solution.

CRS is clinically observed in cases where large numbers of lymphocytes (B cells, T cells, and/or natural killer cells) and/or myeloid cells (macrophages, dendritic cells, and monocytes) become activated and release inflammatory cytokines including IL-1beta, TNFalpha, IFNbeta, IFNgamma, IL-6, and IL-8. CRS is caused by a hyperactivated T-cell response which is not tissue specific and thus causes reactivity with normal issue. This results in the production of high levels of CD4 T-helper cell cytokines or increased migration of cytolytic CD8 T cells within normal tissues. Weber, J. S., et al., “Toxicities of Immunotherapy for the Practitioner,” Journal of Clinical Oncology, 33, no. 18 (June 2015) 2092-2099. The onset of symptoms may occur within a period of minutes to hours after administration of an IO therapy. Timing of symptom onset and CRS severity may depend on the inducing agent and the magnitude of the resulting immune cell activation. CRS can lead to serious organ damage and failure; such injury includes pulmonary infiltrates, lung injury, acute respiratory distress syndrome, cardiac dysfunction, cardiovascular shock, neurologic toxicity, disseminated intravascular coagulation (DIC), hepatic failure, or renal failure.

CRS has been reported following the administration of IO therapies including HSCT, cancer vaccines (either alone or in combination with adoptive T-cell therapy), mAbs, and CAR-T cells. CRS is a potentially life-threatening toxicity, with some patients requiring extensive intervention and life support. Patients have experienced neurological damage and/or death. Diagnosis and management of CRS in response to immune cell-based therapies is routinely based on clinical parameters and symptoms. Lee et al. has described a revised CRS grading system, shown below in Table 3. Lee, D. et al. (2014) Blood 124(2): 188-195.

TABLE 3

| Grade | Toxicity |
|---|---|
| Grade 1 | Symptoms are not life threatening and require symptomatic |
| | treatment only, e.g., fever, nausea, fatigue, headache, myalgias, |
| | malaise |
| Grade 2 | Symptoms require and respond to moderate intervention |
| | Oxygen requirement <40% or |
| | Hypotension responsive to fluids or low dose of one vasopressor |
| | or Grade 2 organ toxicity |
| Grade 3 | Symptoms require and respond to aggressive intervention |
| | Oxygen requirement ≥40% or |
| | Hypotension requiring high dose or multiple vasopressors |
| | or Grade 3 organ toxicity or grade 4 transaminitis |
| Grade 4 | Life-threatening symptoms |
| | Requirement for ventilator support or |
| | Grade 4 organ toxicity (excluding transaminitis) |
| Grade 5 | Death |

Grades 2-4 refer to CTCA.E v4.0 grading

Standard treatment involves vigilant supportive care and treatment with immunosuppressive drugs (e.g., anti-cytokine antibodies such as tocilizumab and corticosteroids). Management of CRS must be balanced with ensuring the efficacy of IO treatments. While early and/or aggressive immunosuppression may mitigate CRS, it may also limit the efficacy of the therapy. There have been reports that CRS may actually be necessary for effective treatment. The goal of CRS management is not to completely suppress it, but to prevent life-threatening toxicity while maximizing any antitumor effects. Lee, D. et al. (2014) Blood 124(2): 188-195.

Immuno-Oncology Therapy

The present disclosure relates particularly to methods of improving the safety of immuno-oncology (IO) treatments while maintaining efficacy. Cancer or autoimmune disease may be viewed as the result of a dysfunction of the normal immune system. The goal of IO is to utilize a patient's own immune system to effect treatment of a disorder. IO treatments may include hematopoietic stem cell transplantation (HSCT), cancer vaccines, monoclonal antibodies (mAbs), and adoptive T-cell immunotherapy

### Examples of Combination Therapies

Examples of therapeutic agents that can be used in combination dosing regimens are described below.

Agents that Target Metabolic Pathways

The second or additional therapeutic agent may target a metabolic pathway different from the pathway targeted by the primary therapeutic agent. For example, the second agent may inhibit a glutaminase, the PI3K pathway, or orotidine 5′-monophosphate (OMP) decarboxylase.

CAR T-Cell Therapy

Adoptive T-cell immunotherapy may be performed with either natural T-cells or with engineered T-cells. Engineered T-cells can include T-cells which have been engineered to express chimeric antigen receptors (CARs) on their surface (CAR-T cells).

Autologous adoptive cell transfer involves the collection, modification, and return of a patient's immune cells, offering a promising immunotherapeutic approach for the treatment of different types of cancers. Typically, leukocytes are isolated, usually by well established density barrier centrifugation, and T lymphocytes are expanded ex vivo using cell culture methods, often relying on the immunomodulatory action of interleukin-2. Once expanded, the cells are administered intravenously to the patent in an activated state. Such cells are referred to as effector T cells. In addition, a combination of anti-CD3 and anti-CD28 antibodies may be used as a surrogate for antigen presentation with appropriate co-stimulation cues to promote the proliferation of T cells in culture.

For T cells, engagement of the CD4⁺ and CD8⁺ T cell receptor (TCR) alone is not sufficient to induce persistent activation of resting naive or memory T cells. Fully functional, productive T cell activation requires a second co-stimulatory signal from a competent antigen-presenting cell (APC).

Co-stimulation is achieved naturally by the interaction of CD28, a co-stimulatory cell surface receptor on T cells, with a counter-receptor on the surface of the APC, e.g., CD80 and/or CD86. An APC may also be used for the antigen-dependent activation of T cells. To induce functional activation rather than toleragenic T cells, APCs must also express on their surface a co-stimulatory molecule. Such APCs are capable of stimulating T cell proliferation, inducing cytokine production, and acting as targets for cytolytic T lymphocytes (CTL) upon direct interaction with the T cell.

Recently, T cells have been genetically engineered to produce artificial T cell receptors on their surface called chimeric antigen receptors (CARs). CARs allow T cells to recognize a specific, pre-selected protein, or antigen, found on targeted tumor cells. CAR-T cells can be cultured and expanded in the laboratory, then re-infused to patients in a similar manner to that described above for adoptive transfer of native T cells. The CAR directs the CAR T-cell to a target cell expressing an antigen to which the CAR is specific. The CAR T cell binds the target and through operation of a stimulatory domain activates the CAR T-cell. In some embodiments, the stimulatory domain is selected from CD28, OX40, CD27, CD2, CD5, ICAM-1, LFA-1 (CD11a/CD18), 4-1BB, or a combination thereof.

CARs may be specific for any tumor antigen. In some embodiments, a CAR comprises an extracellular binding domain specific for a tumor antigen. In some embodiments, a tumor antigen is selected from TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-llRa, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGFI receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In some embodiments, a CAR comprises an extracellular binding domain specific for a tumor targeting antibody. In some embodiments, an extracellular binding domain specific for a tumor targeting antibody binds an Fc portion of a tumor targeting antibody. In some embodiments, an extracellular binding domain specific for a tumor targeting antibody comprises an Fc receptor or an Fc binding portion thereof. In some embodiments, an Fc receptor is an Fc-gamma receptor, an Fc-alpha receptor, or an Fc epsilon receptor. In some embodiments, an extracellular binding domain can be an extracellular ligand-binding domain of CD 16 (e g., CD16A or CD16B), CD32 (e g., CD32A, or CD32B), or CD64 (e g., CD64A, CD64B, or CD64C).

In some embodiments, a CAR comprises a transmembrane domain. In some embodiments, a transmembrane domain is selected from CD8α, CD8β, 4-1BB, CD28, CD34, CD4, FccRIγ, CD16 (e g., CD16A or CD16B), OX40, CD3ζ, CD3ε, CD3γ, CD3δ, TCRα, CD32 (e g., CD32A or CD32B), CD64 (e g., CD64A, CD64B, or CD64C), VEGFR2, FAS, and FGFR2B, or a combination thereof. In some embodiments, the transmembrane domain is not CD8α. In some embodiments, a transmembrane domain is a non-naturally occurring hydrophobic protein segment.

In some embodiments, a CAR comprises a co-stimulatory domain for T-cell activation. In some embodiments, a co-stimulatory domain is selected from CD28, OX40, CD27, CD2, CD5, ICAM-1, LFA-1 (CD11a/CD18), 4-1BB, GITR, HVEM, TIM1, LFA1, or CD2, a functional fragment thereof, or a combination thereof. In some embodiments, a CAR comprises two or more co-stimulatory domains. In some embodiments, the two or more co-stimulatory domains are selected from CD28, OX40, CD27, CD2, CD5, ICAM-1, LFA-1 (CD11a/CD18), 4-1BB, GITR, HVEM, TIM1, LFA1, or CD2.

Cytokine release syndrome (CRS) is a common and potentially lethal complication of CAR-T cell therapy. It is a non-antigen specific toxicity that can occur as a result of the high-levels of CAR-T cell expansion and immune activation typically required to mediate clinical benefit using modern immunotherapies such as CAR-T cell transfer. Timing of symptom onset and CRS severity depends on the inducing agent and the magnitude of immune cell activation. Symptom onset typically occurs days to occasionally weeks after T cell infusion, coinciding with maximal in vivo T-cell expansion.

The incidence and severity of CRS following CAR-T therapy for cancer has recently been reported to be greater in patients having large tumor burdens. Without wishing to be bound by any theory, it is believe that this is due to the expression of production of pro-inflammatory cytokines such as TNF-α by the adoptively transferred expanding and activated CAR-T cell populations. CRS following CAR-T therapy has been consistently associated with elevated IFNγ, IL-6, and TNF-α levels, and increases in IL-2, granulocyte macrophage-colony-stimulating factor (GM-CSF), IL-10, IL-8, IL-5, and fracktalkine have also been reported.

Cancer Vaccines

In some embodiments an immune-oncology therapy is a cancer vaccine. A cancer vaccine is an immunogenic composition which stimulates a patient's immune system to produce anti-tumor antibodies, thereby enabling the immune system to target and destroy cancerous cells. In some embodiments, a cancer vaccine is a peptide vaccine. In some embodiments, a cancer vaccine is a conjugate vaccine.

In some embodiments, a cancer vaccine is used in combination with adoptive T cell therapy. In some embodiments, a cancer vaccine is administered to a patient, after which tumor specific T cells are obtained from the patient, isolated, expanded ex vivo, and then administered to the patient. In some embodiments, the ex vivo expansion of tumor specific T cells provides for a method of obtaining a greater number of T cells which may attack and kill cancerous cells than what could be obtained by vaccination alone. In some embodiments, adoptive T cell therapy comprises culturing tumor infiltrating lymphocytes. In some embodiments, one particular T cell or clone is isolated and expanded ex vivo prior to administration to a patient. In some embodiments, a T cell is obtained from a patient who has received a cancer vaccine.

Administration of cancer vaccines, either alone or in combination with adoptive T cell transfer has been reported to result in CRS.

Human Stem Cell Transplantation (HSCT)

HSCT is the transplantation of stem cells to reestablish hematopoietic function in a patient with defective bone marrow or immune system. In some embodiments, the stem cells are autologous. In some embodiments, the stem cells are allogeneic. In some embodiments the transplant is performed by intravenous infusion.

In some embodiments, autologous HSCT may be used to treat multiple myeloma, non-Hodgkin lymphoma, Hodgkin disease, acute myeloid leukemia, neuroblastoma, germ cell tumors, autoimmune disorders (e.g., systemic lupus erythematosus [SLE], systemic sclerosis), or amyloidosis.

In some embodiments, allogeneic HSCT may be used to treat acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, Epidermolysis Bullosa, severe congenital neutropenia, Shwachman-Diamond syndrome, Diamond-Blackfan anemia, or leukocyte adhesion deficiency.

In some embodiments, stem cells are obtained from a donor for administration to a patient. In some embodiments, the donor is an identical twin of the patient. In some embodiments, the donor is a matched donor related to the patient. In some embodiments, the donor is a matched donor unrelated to the patient. In some embodiments, the donor is a mismatched donor related to the patient. In some embodiments, the donor is haploidentical to the patient.

In some embodiments stem cells are obtained from bone marrow, peripheral blood, or umbilical cord blood.

HSCT may result in graft vs. host disease (GvHD), which remains a major cause of morbidity and mortality in patients undergoing HSCT. Even though there have been advances in prevention and post-transplant immunosuppressive strategies, it is estimated that 20-50% of all HSCT patients will experience at least moderate GvHD. Inflammatory cytokine release, e.g., CRS, is likely the primary mediator of acute GvHD, and activation of T-cells is one step in this complex process. Ball, L. M. & Egeler, R. M., “Acute GvHD: pathogenesis and classification,” Bone Marrow Transplantation (2008) 41, S58-S64. Bouchlaka, M. N., “Immunotherapy following hematopoietic stem cell transplantation: potential for synergistic effects,” Immunotherapy. 2010 May; 2(3): 399-418.

Monoclonal Antibodies (mAbs)

Monoclonal antibodies are useful in the treatment of various cancers. mAb cancer treatments utilize natural immune system functions to attack cancerous cells. Administration of mAbs specific for tumor antigens can be useful in targeting the tumor cells for destruction by the immune system. In some cases mAbs can trigger lysis of cancer cells, block cancer cell growth/replication, prevent angiogenesis, act as checkpoint inhibitors, and in some cases act to bind a tumor antigen while also activating specific immune cells. In some embodiments, a monoclonal antibody is monospecific. In some embodiments, a monoclonal antibody is bispecific. In some embodiments, a monoclonal antibody is a checkpoint inhibitor. In some embodiments, a mAb may be used in combination with CAR-T therapy.

When activated by therapeutic monoclonal antibodies, T-cell surface receptors can cause CRS. In some embodiments, antibodies which may induce CRS include anti-CD3 antibodies, anti-CD20 antibodies, anti-CD28 antibodies, anti-CTLA-4 antibodies, anti-PD-1 antibodies, and anti-PD-L1 antibodies. In some embodiments, antibodies which may induce CRS include alemtuzumab, muromonab-CD3, rituximab, tosituzumab, CP-870,893, LO-CD2a/BTI-322, TGN1412, pembrolizumab, nivolumab, and ipilimumab.

### EXAMPLES

### Example 1: Determining Brequinar Levels in Plasma

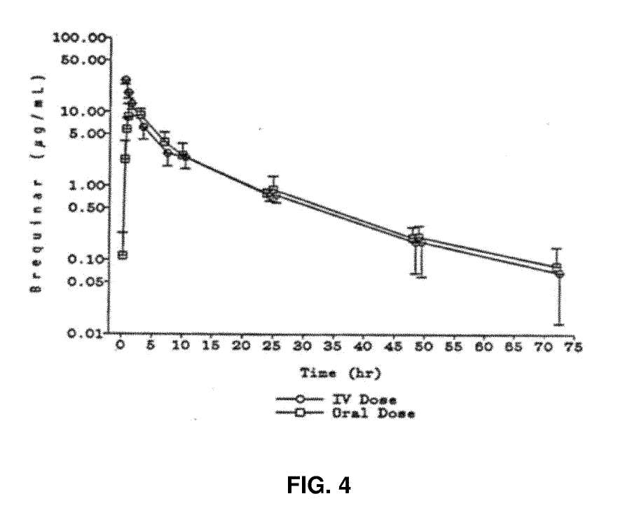

FIG. 4 is a scatter plot illustrating the concentration of brequinar in subject plasma over time when administered twice weekly.

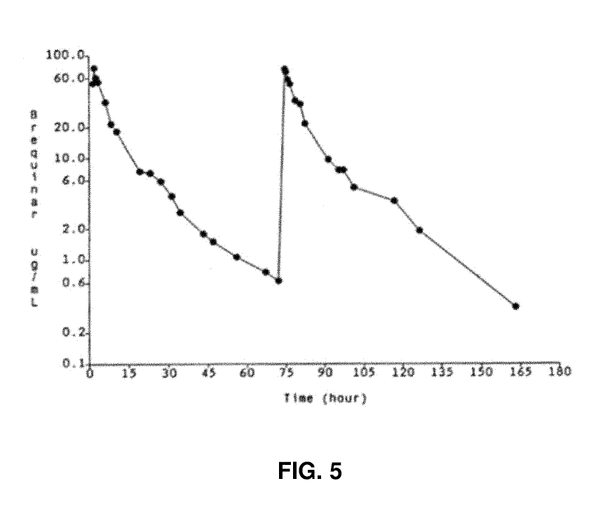

FIG. 5 is a scatter plot illustrating the bioavailability of an IV formulation of brequinar as compared to an oral dosage form.

The concentration of DHO in a subject's plasma is correlated with the concentration of DHODH inhibitor in the plasma. As provided herein, the disclosed methods provide, in some embodiments, administering the DHODH inhibitor when the DHO concentration in the plasma is either at least a particular efficacy threshold or below a potential toxic threshold (i.e., a pre-determined level).

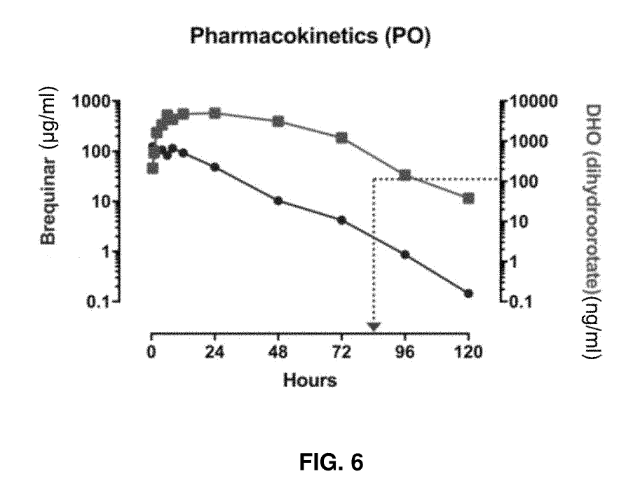

FIG. 6 is a scatter plot illustrating the concentration of brequinar in mice at a dose of 50 mg/kg over time. The dashed line illustrates that about 100 ng/mL concentration of DHO remains in the plasma at about 84 hours.

### Example 2: Adverse Events Observed in Subjects Receiving Brequinar

Brequinar was administered intravenously to 209 subjects once a week with a median number of doses per patient of 4 (range 1 to 24) at a median dose of 1200 mg/m² (range 588 to 3110). Adverse events that were observed in more than 3% of subjects are reported in Table 4, below:

TABLE 4

| | | | No. of Patients Experiencing | | | |
|---|---|---|---|---|---|---|
| | No. of | | the AE, 5y Max Grade | | | |
| | Patients | Percent | 1 | 2 | 3 | 4 |
| All Body Systems | 202 | 95.7 | 36 | 76 | 55 | 35 |
| Thrombocytopenia | 94 | 45.0 | 26 | 31 | 16 | 21 |
| Nausea | 91 | 43.5 | 59 | 19 | 12 | 1 |
| Anemia | 90 | 43.1 | 14 | 48 | 23 | 5 |
| Diarrhea | 77 | 36.8 | 43 | 21 | 10 | 3 |
| Vomit | 73 | 34.9 | 32 | 24 | 12 | 5 |
| Leukopenia | 69 | 33.0 | 26 | 31 | 10 | 2 |
| Stomatitis | 60 | 28.7 | 32 | 20 | 7 | 1 |
| Rash | 53 | 25.4 | 26 | 15 | 9 | 3 |
| Mucositis | 52 | 24.9 | 23 | 15 | 11 | 3 |
| Granulocytopenia | 37 | 19.6 | 16 | 17 | 3 | 5 |
| Fatigue | 33 | 15.8 | 23 | 8 | 2 | 0 |
| Pain Inject Site | 24 | 11.5 | 24 | 0 | 0 | 0 |
| Anorexia | 15 | 7.2 | 11 | 3 | 1 | 0 |
| Fever | 11 | 5.3 | 4 | 7 | 0 | 0 |
| Constipation | 10 | 4.8 | 6 | 2 | 1 | 0 |
| Somnolence | 9 | 4.3 | 7 | 2 | 0 | 0 |
| Pain, Abdominal | 8 | 3.8 | 4 | 3 | 1 | 0 |
| Dyspepsia | 7 | 3.3 | 6 | 1 | 0 | 0 |
| Headache | 7 | 3.3 | 4 | 3 | 0 | 0 |
| Infection | 7 | 3.3 | 4 | 3 | 0 | 0 |

### Example 3: Determining DHO Levels in Plasma Samples Using DHO as a Standard

Prior to analysis the plasma samples are deproteinized by centrifugation through a 50 kD Amicon ultrafilter. 10 μL of a plasma sample is spiked with 5 μL of a standard solution of (S)-4,5-dihydroorotic-4,5,6-carboxy-¹³C4 acid (¹³C4-DHO) and then diluted with 35 μL of 0.1% (w/w) formic acid. Samples are injected into a reverse-phase 4 μm C18 column (Synergy Hydro RP-80A, 3 μm, 150×3 mm; Phenomenex, Australia). Chromatography is performed at 30° C. with a total flow rate of 0.3 mL/min, using solvent A (aqueous 5 mM ammonium acetate, 0.05% (w/v) formic acid) and solvent B (0.05% (w/v) formic acid in methanol) in a linear gradient elution from A:B 98:2 (v/v) to 85:15 (v/v) over 11 minutes, the 40:60 (v/v) for 1 minute, before returning to initial conditions for a further 6 minutes of equilibration.

Tandem mass spectrometry (LC/MS/MS) is performed using an Applied Biosystems API 4000 QTRAP mass spectrometer equipped with a Turbo-V-Spray source with the gas temperature set at 500° C. The source operated an electrospray interface (ESI) with switching ionization polarity (between +5000 V and −4000 V) during the run (18 min). The eluent is monitored by specific ion transitions for DHO and the internal standard. All data is quantified using Applied Biosystems software.

### Example 4: Determining DHO Acid Levels in Plasma Samples Using Orotic Acid as a Standard

Prior to analysis the plasma samples are deproteinized by centrifugation through a 50 kD Amicon ultrafilter. 10 μL of a plasma sample is spiked with 5 μL of a standard solution of 15N2-orotic acid and then diluted with 35 μL of 0.1% (w/w) formic acid. Samples are injected into a reverse-phase 4 μm C18 column (Synergy Hydro RP-80A, 3 μm, 150×3 mm; Phenomenex, Australia). Chromatography is performed at 30° C. with a total flow rate of 0.3 mL/min, using solvent A (aqueous 5 mM ammonium acetate, 0.05% (w/v) formic acid) and solvent B (0.05% (w/v) formic acid in methanol) in a linear gradient elution from A:B 98:2 (v/v) to 85:15 (v/v) over 11 minutes, the 40:60 (v/v) for 1 minute, before returning to initial conditions for a further 6 minutes of equilibration.

Tandem mass spectrometry (LC/MS/MS) is performed using an Applied Biosystems API 4000 QTRAP mass spectrometer equipped with a Turbo-V-Spray source with the gas temperature set at 500° C. The source operated an electrospray interface (ESI) with switching ionization polarity (between +5000 V and −4000 V during the run (18 min). The eluent is monitored by specific ion transitions for DHO and the internal standard. All data was quantified using Applied Biosystems SCIEX Multiquant software.

### Example 5: Determined DHO Levels in Healthy Subjects and Cancer Patients

The concentration of dihydroorotic acid in human K2EDTA plasma samples was determined by reversed-phase high performance liquid chromatography with tandem mass spectrometric detection (LC-MS/MS). Plasma samples (50 μL) were spiked with 5 μL of a 1.0 μg/mL solution of (S)-4,5-dihydroorotic-4,5,6,carboxy-13C4 acid (13C4-DHO) in water, which was used as the internal standard (IS), then vigorously mixed with acetonitrile (200 μL) for 5 min. After centrifugation (12,000 rpm, 5 min), 150 μL of the supernatant was applied to a preconditioned Waters (Milford, Mass.) Oasis MAX solid phase extraction cartridge (1 cc, 30 mg). The cartridge was washed sequentially with water and methanol before eluting the analyte with 1% (v/v) formic acid in methanol (1 mL). The eluent was evaporated under a stream of nitrogen and reconstituted in 50 μL of 1% (v/v) formic acid in water. The solution was transferred into a conical bottom insert placed in an amber autosampler vial and sealed. A 10 μL aliquot of the solution was injected onto a Phenomenex (Torrance, Calif.) Synergi 4 μm Hydro-RP 80A HPLC column (250 mm×3.0 mm i.d.) preceded by an AQ C18 guard cartridge (4.0 mm×3.0 mm i.d.) and separated using an isocratic mobile phase composed of 0.05% (v/v) formic acid in water at a flow rate of 0.5 mL/min. An Agilent Technologies (Santa Clara, Calif.) model G6410B triple quadrupole mass spectrometer with an electrospray ionization interface was used for detection. Nitrogen was used as the nebulizing gas (30 p.s.i.) and drying gas (10 L/min, 350° C.). With a transfer capillary potential of 1,500 V, negative ions resulting from the m/z 157→113 transition for dihydroorotic acid and the m/z 161→117 transitions for the IS were measured by multiple reaction monitoring (dwell time, 150 msec; fragmentor potential, 70 V; collision energy, 4 V; collision cell accelerator voltage, 4 V). Quantitation was based upon integrating the extracted ion chromatograms for both transitions to provide peak areas and calculating the ratio of the analyte peak area to the IS peak area for each sample.

Table 5 provides data of DHO concentration for samples from certain random cancer patients, samples from healthy subjects, and samples from mice.

TABLE 5

| | | | |
|---|---|---|---|
| | | ASSAY DHO | AVG. ASSAY |
| Subject No. | Sample | CONC. ng/mL | CONC. ng/mL |

Cancer Patients

| | | | |
|---|---|---|---|
| 1 | 1 | 4.1 | |
| | 2 | 4.25 | 4.18 |
| 2 | 1 | 0 | |
| | 2 | 0 | 0.00 |
| 3 | 1 | 1.17 | |
| | 2 | 0.19 | 0.68 |
| 4 | 1 | 15.1 | |
| | 2 | 15.4 | 15.25 |
| 5 | 1 | 5.2 | |
| | 2 | 5.3 | 5.25 |
| 6 | 1 | 0.41 | |
| | 2 | 0.86 | 0.64 |

Healthy Subjects

| | | | |
|---|---|---|---|
| 1 | 1 | 0 | |
| | 2 | 0 | 0.00 |
| 2 | 1 | 0 | |
| | 2 | 0 | 0.00 |
| 3 | 1 | 0 | |
| | 2 | 0 | 0.00 |
| 4 | 1 | 0 | |
| | 2 | 0 | 0.00 |
| 5 | 1 | 0 | |
| | 2 | 0 | 0.00 |
| 6 | 1 | 0 | |
| | 2 | 0 | 0.00 |

Mice

| | | | |
|---|---|---|---|
| 1 | 1 | 1 | 1 |
| | 2 | 0.06 | 0.00 |

Table 6 provides patient data for 20 anonymous cancer patients whose DHO acid concentration was measured.

TABLE 6

| | | | | | | | | Immio- | Immio- | |
|---|---|---|---|---|---|---|---|---|---|---|
| | Diag- | | | | Form and | Chemo- | Blast Cells by | phenotyping | phenotyping | |
| No. | nosis | Sample | Gender | Age | Stage | therapy | Morphology* | CD34⁺* | CD19⁺/CD5⁺* | Cytogenetics |
| 1 | AML | Blood | F | 60 | M0 or M5a | | | 12.6 (BM) | | 45,XX, |
| | | & | | | | | | | | −3,der(5)t(5;3)(q13;q12), |
| | | Marrow | | | | | | | | −7,inv(12)(p 11,2q24.1), |
| | | | | | | | | | | dic( 13;22)(p 12;p |
| | | | | | | | | | | 12),+1~2mar[8]/46, |
| | | | | | | | | | | XX1121 |
| 2 | AML | | | | | | | | | |
| 3 | AML | Blood | M | 84 | Untreated | | 30-40 (BM) | 1.64 (PB)/43.1 (BM) | | |
| 4 | AML | | | | | | | | | |
| 5 | AML | | | | | | | | | |
| 6 | AML | Blood | M | 35 | | Tretinoin | 65 (PB)/43 (BM) | 39 (PB) | | |
| 7 | AML | Blood | F | 37 | M3 | Tretinoin | 75 (PB)/79 (BM) | 0.1 | | |
| | | | | | | Idarubicin | | | | |
| | | | | | | Arsenic | | | | |
| | | | | | | trioxide | | | | |
| 8 | AML | Blood | M | 68 | | | 60 (BM) | 11 (PB) | | |
| 9 | AML | Blood | M | 70 | | | 76 (BM) | 97 (PB) | | ish(D7Z1x2, |
| | | | | | | | | | | D7S486x1)[41/200],(KAT |
| | | | | | | | | | | 6Ax3)[46 1/500],(D8Z2, |
| | | | | | | | | | | MYC)x3 [186/200], |
| | | | | | | | | | | (RLINX1T1x3)[461/5001 |
| 10 | AML | Blood | F | 57 | Relapsed | Retinoic | 0 (PB)/11 (BM) | 0.7 (PB) | | t(15;17) PML/RARA |
| | | & | | | | acid, | | | | fusion [by FISH]) |
| | | Marrow | | | | Arsenic, | | | | Abnormal 918″ |
| | | | | | | Idarubicin, | | | | |
| | | | | | | Arsenic | | | | |
| 11 | AML | Blood | M | 65 | non | | 38 (BM) | 0.77 (PB) | | FLT3/NPM1 mutations |
| | | | | | promyelocytic | | | | | |
| | | | | | with monocytic | | | | | |
| | | | | | differentiation | | | | | |
| 12 | CLL | Blood | M | 53 | | | | | 97 (PB)/91 | |
| | | & | | | | | | | (BM) | |
| | | Marrow | | | | | | | | |
| 13 | CLL | Blood | M | 75 | Relapsed | | | | 85 (PB)/75 | 7.5% have del[13q/14]- |
| | | | | | | | | | (BM) | specific signal |
| 14 | CLL | Blood | F | 56 | Relapsed | Rituxan | | | 27.7 (PB)/67.5 | |
| | | & | | | refractory | | | | (BM) | |
| | | Marrow | | | | | | | | |
| 15 | CLL | Blood | F | 67 | Relapsed | | | | 53.4 (PB)/61.4 | |
| | | & | | | | | | | (BM) | |
| | | Marrow | | | | | | | | |
| 16 | CLL | Blood | F | 69 | | | | | 3.73 (PB) | |
| 17 | CML | | | | | | | | | |
| 18 | CML | Blood | M | 50 | Newly | | | 0.8 (PB)/1.4 (BM) | | BCR-ABL positive |
| | | & | | | Diagnosed, | | | | | |
| | | Marrow | | | Chronic Phase | | | | | |
| 19 | CML | Blood | M | 31 | Relapsed | BCR-ABL, | | 0.72 (PB)/7.1 (BM) | | |
| | | & | | | refractory | Gleevec | | | | |
| | | Marrow | | | | | | | | |
| 20 | CML | Blood | M | | Newly | N/A | | 1.6 (PB)/1.8 (BM) | | BCR-ABL positive |
| | | & | | | diagnosed | | | | | |
| | | Marrow | | | chronic phase | | | | | |

* (PB = % Blood, BM % Marrow)

Table 7 provides baseline endogenous DHO acid concentration in plasma samples from the set of 20 cancer patients.

TABLE 7

| No. | Assay 1 | Assay 2 | Assay 3 | Mean |
|---|---|---|---|---|
| 1 | <LOD | <LOD | | <LLQ |
| 2 | 13.8 | 15.2 | | 14.5 |
| 3 | 58.1 | 49.0 | | 53.6 |
| 4 | 32.8 | 30.0 | | 31.4 |
| 5 | <LOD | <LLQ | | <LLQ |
| 6 | 9.5 | 8.4 | | 8.99 |
| 7 | <LLQ | <LLQ | | <LLQ |
| 8 | 18.0 | 16.4 | | 17.2 |
| 9 | 6.7^{b} | 33.4 | 29.9 | 31.6 |
| 10 | 12.8 | 13.9 | | 13.4 |
| 11 | 17.0^{b} | 11.8 | 10.2 | 11.0 |
| 12 | <LOD | <LOD | | <LLQ |
| 13 | <LOD | <LOD | | <LLQ |
| 14 | <LOD | <LOD | | <LLQ |
| 15 | 6.51 | 5.14 | | 5.83 |
| 16 | <LLQ | <LLQ | | <LLQ |
| 17 | 37.1^{b} | <LOD | <LOD | <LLQ |
| 18 | <LOD | <LLQ | | <LLQ |
| 19 | <LOD | <LOD | | <LLQ |
| 20 | 5.1^{b} | <LLQ | <LLQ | <LLQ |

^{a}<LOD, below the limit of detection (analyte peak not distinguishable from baseline); <LLQ, assayed concentration below the lower limit of quantitation (5.0 ng/mL).
^{b}Result not used for calculation of the mean assayed concentration and percent difference.

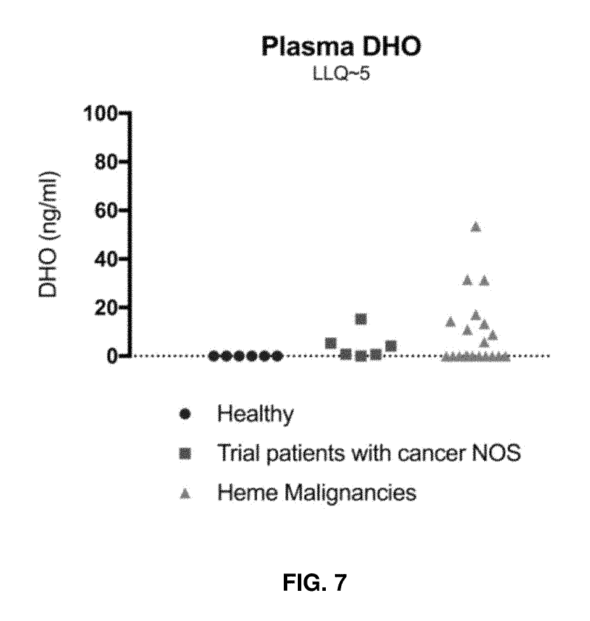

FIG. 7 is a scatter plot illustrating the baseline DHO levels in random cancer patients and healthy patients, as reported in Table 5.

### Example 6: Clinical Dosing Regimens Previously Tested for Brequinar in Patients with Refractory Solid Tumors

Previous clinical dosing regimens assessed brequinar for use in treating refractory solid tumors in patients. For example, Arteaga reported administration of brequinar as “single daily i.v. bolus over a 5-day period repeated every 28 days.” Arteaga, et al., “Phase I clinical and pharmacokinetic trial of Brequinar sodium (DuP 785; NSC368390),” Cancer Res., 49(16):4648-4653 (Aug. 15, 1989). Specifically, Arteaga administered “one hundred seven courses of treatment at dosages ranging from 36 to 300 mg/m²/day×5” to 45 patients (31 male and 14 female) with refractory solid tumors. The reported median age of these patients was 58 years (range 30-74); and the median Southwest Oncology Group performance status was reported to be 1 (range, 0-3). Arteaga found “for the daily×5 i.v. schedule, the recommended dose of Brequinar for phase II evaluation is 250 mg/m² for good risk patients and 135 mg/m² for poor risk patients.” Burris reported “investigating the pharmacokinetic and toxicity of brequinar in combination with cisplatin” where patients were initially treated with weekly brequinar, in combination with an every-three-week administration of cisplatin. See Burris, et al., “Pharmacokinetic and phase I studies of brequinar (DUP 785; NSC368390) in combination with cisplatin in patients with advanced malignancies,” Invest. New Drugs, 16(1):19-27 (1998). Burris found that “due to toxicity, the schedule was modified to a 28-day cycle with brequinar given on days 1, 8, 15, and cisplatin on day 1.” A total of 24 patients (16 male, 8 female; median age 57; median performance status 1) received 69 courses of therapy. Six dose levels were explored, with cisplatin/brequinar doses, respectively, of 50/500, 50/650, 50/860, 60/860, 75/650, and 75/860 mg/m². Burris concluded that “full dose of 75 mg/m² cisplatin (day 1) can be administered with 650 mg/m² brequinar (days 1, 8 and 15) without significant modifications of individual drug pharmacokinetic parameters.” Noe reported “in vitro and in vivo studies [of brequinar] demonstrate the superiority of prolonged drug exposure in achieving tumor growth inhibition. This phase I study evaluated the administration of brequinar sodium by short, daily i.v. infusion for 5 days repeated every 4 weeks.” See Noe, et al., “Phase I and pharmacokinetic study of brequinar sodium (NSC368390),” Cancer Res., 50(15):4595-4599 (1990). Noe examined “fifty-four subjects . . . received drug in doses ranging from 36-300 mg/m².” Noe found that “the maximum tolerated dose on the ‘daily times 5’ schedule was 300 mg/m²” and that “the recommended phase II dose is 250 mg/m².” Noe concluded that “pharmacodynamic analysis of the day 1 kinetic parameters and the toxicities occurring during the first cycle of drug therapy revealed significant correlations between mucositis and dose, AUC, and peak brequinar concentration; between leukopenia and AUC and peak drug concentration; and between thrombocytopenia and beta elimination rate.”

Schwartsmann reported dosing brequinar in 43 patients who “received 110 courses of Brequinar sodium by short-term intravenous (i.v.) infusion” every 3 weeks.” See Schwartsmann, et al., “Phase I study of Brequinar sodium (NSC 368390) in patients with solid malignancies,” Cancer Chemother. Pharmacol., 25(5):345-351 (1990). Schrwatsmann based dose escalation on “a modified Fibonacci scheme,” initially, but relied on a pharmacologically guided dose escalation after PK data became available, noting that “at toxic levels, dose escalation was applied on the basis of clinical judgement.” Swchwartsmann reported that “[t]he maximum tolerable doses for poor- and good-risk patients were 1,500 and 2,250 mg/m², respectively. One mixed response was observed in a patient with papillary carcinoma of the thyroid. The recommended doses for phase II studies are 1,200 and 1,800 mg/m² Brequinar sodium, given by a 1-h i.v. infusion every 3 weeks to poor- and good-risk patients, respectively.”

### Example 7: Exemplary Clinical Dosing in Accordance with the Present Disclosure Inclusion Criteria

The following are proposed inclusion criteria for subjects in a proposed clinical trial:
Willing and able to provide written informed consent for the trial.
Adults, 18 years of age and older, with pathologically confirmed, relapsed or refractory acute myelogenous leukemia.
≥18 years of age on day of signing informed consent
ECOG Performance Status 0 to 2.
Cardiac ejection fraction ≥40%
Adequate hepatic function (unless deemed to be related to underlying leukemia)
Direct bilirubin ≤2×ULN
ALT ≤3×ULN
AST ≤3×ULN
Adequate renal function as documented by creatinine clearance ≥30 mL/min based on the Cockcroft-Gault equation

In the absence of rapidly proliferative disease, the interval from prior leukemiadirected therapy to time of study initiation will be at least 7 days for cytotoxic or non-cytotoxic (immunotherapy) agents. Hydrea is allowed up to 48 hours prior to the first dose for patients with rapidly proliferative disease.

The effects of brequinar on the developing human fetus are unknown. For this reason, women of child-bearing potential and men must agree to use adequate contraception (hormonal or barrier method of birth control; abstinence) prior to study entry and for the duration of study participation. Should a woman become pregnant or suspect she is pregnant while she or her partner is participating in this study, she should inform her treating physician immediately. Men treated or enrolled on this protocol must also agree to use adequate contraception prior to the study, for the duration of study participation, and for 90 days after completion of brequinar administration.

Male subjects must agree to refrain from sperm donation from initial study drug administration until 90 days after the last dose of study drug.

### Exclusion Criteria

The following are proposed exclusion criteria for excluding a subject in the study.
White blood count >25×109/L (note: hydroxyurea is permitted to meet this criterion).
Any concurrent uncontrolled clinically significant medical condition, laboratory abnormality, or psychiatric illness that could place the participant at unacceptable risk of study treatment.
QTc interval using Fridericia's formula (QTcF)≥470 msec. Participants with a bundle branch block and prolonged QTc interval may be eligible after discussion with the medical monitor.
The use of other chemotherapeutic agents or anti-leukemic agents is not permitted during study with the following exceptions:
Intrathecal chemotherapy for prophylactic use or maintenance of controlled CNS leukemia.
Use of hydroxyurea may be allowed during the first 2 weeks of therapy if in the best interest of the participant and is approved by the medical monitor.
AML relapse less than 6 months following stem cell transplantation.
Presence of graft versus host disease (GVHD) which requires an equivalent dose of ≥0.5 mg/kg/day of prednisone or therapy beyond systemic corticosteroids (e.g. cyclosporine or other calcineurin inhibitors or other immunosuppressive agents used for GVHD).
Active cerebrospinal involvement of AML.
Diagnosis of acute promyelocytic leukemia (APL)
Clinically active hepatitis B (HBV) or hepatitis C (HCV) infection.
Severe gastrointestinal or metabolic condition that could interfere with the absorption of oral study medication
Prior malignancy, unless it has not been active or has remained stable for at least 5 years. Participants with treated non-melanoma skin cancer, in situ carcinoma or cervical intraepithelial neoplasia, regardless of the disease-free duration, are eligible if definitive treatment for the condition has been completed. Participants with organ-confined prostate cancer with no evidence of recurrent or progressive disease are eligible if hormonal therapy has been initiated or the malignancy has been surgically removed or treated with definitive radiotherapy.
Nursing women or women of childbearing potential (WoCBP) with a positive urine pregnancy test.

### Dose Levels

Proposed dosing levels are provided below:

Patients are dosed every 3.5 days. An example schedule of events is reported in Table 8.

TABLE 8

| | | | | | | | | | | | F/U | |
|---|---|---|---|---|---|---|---|---|---|---|---|---|
| | | | | | | | Dose Escalation | | Maintenance | | Phone | |
| | | | | | | | Cycle (Cycle | | Dose (no dose | | Call | |
| | | Cycle 1 (Study Days 1-14) | | | | | 2 and beyond | | adjustment) | | Final | |
| | | Day | Day | Day | Day | Day | as needed) | | Every 2 weeks | Final | Visit | |
| Procedures^{a} | Screen^{b} | 1 | 2 | 3 | 4 | 8 | Day 1 | Day 8 | Day 1 | Visit | +2 wks | Survival |
| Informed Consent | X | | | | | | | | | | | |
| AE/Concomitant | X | X | X | X | X | X | X | X | X | X | X | |
| Medication Assessment | | | | | | | | | | | | |
| Demographics^{c} | X | | | | | | | | | | | |
| Physical Exam | X | X | | | | | X | | X | X | | |
| (including weight) | | | | | | | | | | | | |
| Vital Signs^{c} | X | X | | | | X | X | X | X | X | | |
| Pregnancy Test^{d} | X | | | | | | | | X | X | | |
| ECOG Performance | X | | | | | | | | | | | |
| Status | | | | | | | | | | | | |
| Hematology/ | X | X | | | | X | X | X | X | | | |
| Chemistry^{e} | | | | | | | | | | | | |
| Chromosomal & | X | | | | | | | | | | | |
| mutational testing^{f} | | | | | | | | | | | | |
| 12-lead ECG | X | | | | | | | | X | X | | |
| MUGA/ | X | | | | | | | | | | | |
| Echocardiogram | | | | | | | | | | | | |
| Bone Marrow Sampling^{g} | X | | | | | | X | | X^{g} | X | | |
| Brequinar/DHO | | X | X | X | X | X | X | X | X | X | | |
| Plasma Sample^{h} | | | | | | | | | | | | |
| Ship Plasma | | | | | | X | | X | X | | | |
| Samples | | | | | | | | | | | | |
| Dispense/Collect | | X | | | | | X | | X | X | | |
| Study Medication | | | | | | | | | | | | |
| Dispense/Collect | | X | | | | | X | | X | X | | |
| Subject Calendar/Diary | | | | | | | | | | | | |
| Survival Assessment | | | | | | | | | | | | X |

^{a}Visit window of ±1 day for dose escalation cycles; window of ±3 days for non-dose-escalation cycles.
^{b}Obtain informed consent prior to performing any screening or study-specific procedures. Screening procedures must be performed within 14 days prior to initial study drug administration. Procedures at C1D1 that are repeats of Screening may be omitted if <72h since Screening assessment.
cDemographic information includes date of birth, height, weight, race, and ethnic origin. Vital signs include heart rate, respiratory rate, seated blood pressure, oral/aural body temperature.
^{d}For women of childbearing potential only.
^{e}CBC differential may be omitted if previous WBC < 0.5 × 10⁹/L
^{f}Per institutional standard of care.
^{g}Local bone marrow sampling (core biopsy and aspirate) will include molecular testing, flow cytometry for minimal residual disease counts (MRD); perform bone marrow sampling at screening, once at C2D8 +/−7 days, at Day 42, and once every 12 weeks after a stable dose has been reached. Only the Day 43 sample will be used to assess hematological toxicity. Ship sample to central lab for future testing. Timing of this procedure may be adjusted to ensure results are available for the next clinic visit.
^{h}Brequinar/DHO plasma sampling schedule: Cycle 1: 0 (pre-dose), post dose 1, 2, 4, 6, 24, 48, 72 hours and C1D8 pre-dose (+84 h after C1D4 dose); Cycle 2 and adjustment cycles: pre-dose Days 1 and 8. Maintenance dose: Day 1 pre-dose. Day 1 PK window ±15 minutes through 6 h draw, window for additional Cl draws ±2 h; window for Cycle 2 and beyond plasma brequinar/DHO draws ±4 h. Plasma samples for brequinar/DHO for expansion cohort are to be obtained prior to dosing on Day 1 of each 2-week cycle.

Another example dosing schema is:

| Dose level | Brequinar (mg/m²) |
|---|---|
| +2 (Target dose) | 800 |
| +2 (Target dose) | 650 |
| 0 (Starting dose) | 500 |
| −1 | 425 |

The dosing sequence (i.e. every 3.5 days) will be subject to revision after review of preliminary efficacy, toxicity, and PK data within this clinical trial. PK data from patients treated at dose level 0 will be used to evaluate the anticipated minimally effective dose, to adjust the dose and schedule, if necessary, in subsequent dose level cohorts.

### Example 8: Determining Analyte Levels in Plasma

The following assay protocol is useful for measuring the concentration of analytes such as pyrazofurin, orotate (i.e., orotate), orotidylate monophosphate (OMP), and uradilyate monophosphate (UMP) in serum samples of subjects.

Prior to analysis 25 μL plasma samples are deproteinized by extraction with a 200 μL of 70:30 acetonitrile:methanol containing 1% formic acid and 1 μg/mL of the internal standard adenosine monophosphate (AMP). The acetonitrile:methanol solution is evaporated at 50° C. with nitrogen and reconstituted with 150 μL of water for injection. Samples are injected into a reverse-phase Waters Atlantis T3 2.1 mm×100 mm, 3 μm column. Chromatography is performed, using solvent A (aqueous 10 mM ammonium acetate, pH 4.8) and solvent B (0.1% (w/v) formic acid in methanol) in a linear gradient elution from A:B 98:2 (v/v) to 85:15 (v/v) over 11 minutes, the 40:60 (v/v) for 1 minute, before returning to initial conditions for a further 6 minutes of equilibration.

Tandem mass spectrometry (LC/MS/MS) is performed using an Applied Biosystems API 5000 QTRAP mass spectrometer equipped with a Turbo-V-Spray source with the gas temperature set at 500° C. The source operated an electrospray interface (ESI) with switching ionization polarity (between +5000 V and −4000 V) during the run (18 min). The eluent is monitored by specific ion transitions for DHO and the internal standard. All data is quantified using Applied Biosystems software.

### Example 9: Concentration of Analyte Associated with Administration of OMP Decarboxylase Inhibitor

An OMP decarboxylase inhibitor, pyrazofurin was administered to mice by oral gavage. The concentration (ng/mL) of analytes selected from pyrazofurin (PYR), orotic acid (i.e., orotate), orotidylate monophosphate (OMP), and uradilyate monophosphate (UMP) in the serum samples were measured according to the assay methods reported in Example 1. The results are reported in Table 9:

TABLE 9

| | PYR 1μM | | | | PYR 0.25 μM | | | | |
|---|---|---|---|---|---|---|---|---|---|
| Source | | Cells | Cells | Cells | Supe* | Cells | Cells | Cells | Supe* |
| Time (hr) | | 1 hr | 4 hr | 24 hr | 24 hr | 1 hr | 4 hr | 24 hr | 24 hr |
| Orotate | ng/ | 339 | 1170 | 1220 | 10005 | 231 | 758 | 1560 | 11300 |
| | mL | | | | | | | | |
| OMP | ng/ | 0 | 10 | 11 | 43 | 0 | 0 | 13 | 49 |
| | mL | | | | | | | | |
| UMP | ng/ | 552 | 408 | 326 | 69 | 737 | 474 | 548 | 67 |
| | mL | | | | | | | | |
| PYR | ng/ | 0 | 0 | 0 | 1010 | 0 | 0 | 0 | 273 |
| | mL | | | | | | | | |

*Supernatant

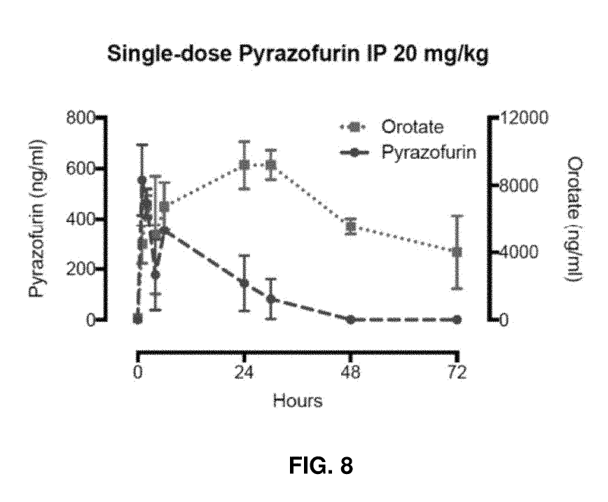

FIG. 8 is a scatter plot illustrating the concentrations of pyrazofurin and orotate in murine plasma over time when pyrazofurin is administered as a single dose (20 mg/kg).

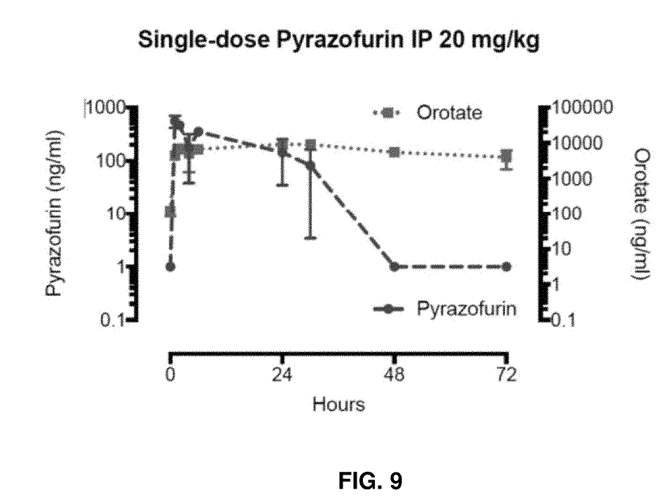

FIG. 9 is a scatter plot illustrating the concentrations of pyrazofurin and orotate in murine plasma over time when pyrazofurin is administered as a single dose (20 mg/kg) on a log scale.

### Example 10: Prior Dosing Regimens

Ohnuma and Holland reported an initial clinical study with pyrazofurin, where twenty-five patients with inoperable carcinoma and lymphoma were given pyrazofurin (PF) “by iv bolus at a dose level ranging from 100 to 300 mg/m² of estimated body surface area.” Further, “five patients with acute leukemia were given [pyrazofurin] by infusion at doses ranging from 250 mg/m²/24 hours to 1500 mg/m²/144 hours.” Ohnuma and Holland found that pyrazofurin “was well tolerated by most patients at doses of 100 mg/m² given as an iv bolus weekly or 250 mg/m² given every 2-3 weeks,” but at infusion of “750 mg/m² given over a period of ˜2-120 hours to leukemic patients resulted in severe but reversible toxicity.” Ohnuma and Holland, “Initial Clinical Study with Pyrazofurin,” Cancer Treatment Reports, 61(3):389-134 (May/June 1977).

Martelo, et al., reported a dosing regimen of administering pyrazofurin “(150 mg/m² by rapid injection) followed 6 hours later by 5-azacytidine (150 mg/m² by continuous infusion for 5 days).” The authors found that “[i]n this study [pyrazofurin] and [5-azacytidine] appeared to have additive toxic effects on skin and mucous membranes at PF doses >50 mg/m².” Specifically, “[t]his toxicity precluded use of [pyrazofurin] at higher doses, which may be important for enhanced uptake of [5-azacytidine] by leukemic cells exposed to PF.” Martelo, et al., “Phase I Study of Pyrazofurin and 5-Azacytidine in Refractory Adult Acute Leukemia,” Cancer Treat. Rep., 65:237-239 (1981).

Gralla, et al., reporting a dosing regimen of administering pyrazofuring “as a rapid iv injection beginning at a weekly dose of 5 mg/kg (200 mg/m²) with increments of 0.5 mg/kg/week (20 mg/m²) until definite but manageable toxicity occurred.” The dosing was adjusted to 4 mg/kg (160 mg/m²) if the wbc count was 3000-3999/microliter or if the platelet count was 75,000-99,000/microliter.” The authors ultimately found that “[m]ajor therapeutic activity did not occur in the patients entered in this trial” and that pyrazofurin “has little therapeutic value as a single agent in this dose schedule in previously treated patients with advanced lung cancer.” Grallo, et al., “Phase II Evaluation of Pyrazofurin in Patients With Carcinoma of the Lung,” Cancer Treat. Rep., 62(3):451-452 (March 1978).

### Example 11: Optimized Dosage Based Metabolite Levels

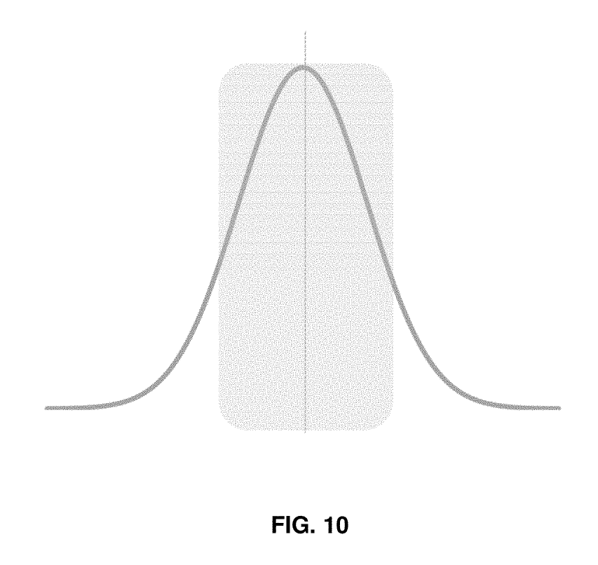

FIG. 10 is a graph showing the therapeutic benefit of a drug, such as brequinar, that targets a metabolic pathway as a function of levels of a metabolite, such as DHO, that is an intermediate in the pathway. On the left side of the graph, levels of the metabolite are below a minimum threshold, and target engagement of the drug is insufficient to have a therapeutic effect. In the grey region of the graph, levels of the metabolite are above a minimum threshold but below a maximum threshold, so the drug has sufficiently engaged its target to provide a therapeutic effect but has not caused effects that are deleterious to healthy cells. On the right side of the graph, levels of the metabolite are above the maximum threshold, and the effects of the drug cause harm to healthy cells. Adjustments to the dosing regimen based on the relationship between therapeutic benefit and metabolite levels are illustrated in Table 10.

TABLE 10

| Metabolite level | Adjustment to dosing regimen |
|---|---|
| below minimum threshold | increase dosage, frequency of dose of |
| | administration, or both |
| above minimum threshold but | no change |
| below maximum threshold | |
| above maximum threshold | decrease dosage, frequency of dose |
| | administration, or both |

### Example 12: Effect of Brequinar-Containing Composition on Patient with AML

The effect of a composition containing brequinar was analyzed on first patient a with acute myeloid leukemia (AML). After administration of a dose of the composition, the patient achieved a DHO plasma level threshold of 1,600 ng/mL in less than 24 hours and remained above that threshold for 84 hours. This patient showed a positive response as indicated by reduction in bone marrow blast count, improvement of extramedullary hematopoiesis, and shift to more differentiation in peripheral blasts.

### Example 13: Effect of Brequinar-Containing Composition on Patient with AML

The effect of a composition containing brequinar was analyzed on second patient a with AML. After administration of a dose of the composition, the patient achieved a DHO plasma level threshold of 2,900 ng/mL in less 24 hours and remained above that threshold for 84 hours. This patient showed a positive response to the disease with a lowering of peripheral blasts and increase in absolute neutrophil count, along with greater differentiation of peripheral blasts.

### Example 14: Effect of Brequinar-Containing Composition on Patient with AML

The effect of a composition containing brequinar was analyzed on second patient a with AML. After administration of a dose of the composition, the patient achieved a DHO plasma level threshold of 133 ng/mL in less than 2 hours and remained above that threshold for 84 hours. This patient showed a positive response as indicated by a trend towards differentiation of his peripheral blasts.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for determining a therapeutically effective dose of an agent to treat a disorder, the method comprising:
receiving information regarding a measured level of a metabolite in a metabolic pathway in a sample from a subject having a disorder;
comparing the received information to a reference that provides an association of a measured level of the metabolite with a recommended dosage adjustment of an agent; and
determining, based on the comparing step, a dosage of the agent that results in the level of the metabolite being raised or maintained above a threshold level, the threshold level being indicative that a sufficient amount of the agent is present in the subject to sufficiently alter the metabolic pathway to ameliorate, reduce, or eliminate at least one sign or symptom of the disorder.

2. The method of claim 1, wherein the recommended dosage adjustment is at least one selected from the group consisting of: increase the dosage by a certain value, decrease the dosage by a certain value, and make no adjustment to the dosage.

3. The method of claim 1, wherein the agent inhibits an enzyme in the metabolic pathway.

4. The method of claim 3, wherein the metabolite is a substrate of the enzyme.

5. The method of claim 1, wherein the metabolic pathway is a nucleotide synthesis pathway.

6. The method of claim 5, wherein the enzyme is selected from the group consisting of aspartate transcarbamoylase, dihydrooratase, dihydroorotate dehydrogenase, orotidine 5′-monophosphate (OMP) decarboxylase, and orotate phosphoribosyl transferase.

7. The method of claim 6, wherein the metabolite is selected from the group consisting of N-carbamoylaspartate, dihydroorotate, orotate, orotidine 5′-monophosphate (OMP), and uridine monophoshpate (UMP).

8. The method of claim 6, wherein the agent comprises one selected from the group consisting of PALA (N-phosphoacetyl-L-aspartate), pyrazofurin, brequinar, a brequinar analog, a brequinar derivative, a brequinar prodrug, a micellar formulation of brequinar, and a brequinar salt.

9. The method of claim 1, wherein the disorder is cancer.

10. The method of claim 9, wherein the cancer is leukemia or prostate cancer.

11. The method of claim 1, further comprising: providing the agent to the subject at the determined dose.

12. A method of determining a therapeutically effective dose of an agent to be provided to a subject to treat a disorder, the method comprising: determining a therapeutically effective dose of an agent based on a measured level of a metabolite in a nucleotide synthesis pathway in a sample from a subject, wherein the therapeutically effective dose of the agent inhibits an enzyme within the nucleotide synthesis pathway to an extent that at least one sign or symptom of the disorder is ameliorated, reduced, or eliminated.

13. The method of claim 12, wherein the nucleotide synthesis pathway is a pyrimidine synthesis pathway.

14. The method of claim 12, wherein the metabolite is a substrate of the enzyme.

15. The method of claim 12, wherein the metabolite is selected from the group consisting of dihydroorotate and orotate.

16. The method of claim 12, wherein the enzyme is selected from the group consisting of dihydroorotate dehydrogenase and orotidine 5′-monophosphate (OMP) decarboxylase.

17. The method of claim 12, wherein the agent comprises one selected from the group consisting of PALA (N-phosphoacetyl-L-aspartate), pyrazofurin, brequinar, a brequinar analog, a brequinar derivative, a brequinar prodrug, a micellar formulation of brequinar, and a brequinar salt.

18. The method of claim 12, wherein the sample is a plasma sample.

19. The method of claim 12, wherein the disorder is a cancer.

20. The method of claim 19, wherein the cancer is leukemia.

21. The method of claim 12, further comprising: providing the agent to the subject at the therapeutically effective dose.

22. A method for assessing in real-time an impact on a tumor of a therapeutic agent, the method comprising:
monitoring, in real-time, a molecule that is associated with a metabolic pathway in a tumor as the molecule moves through the metabolic pathway in the tumor; and
assessing an impact on the tumor of a therapeutic agent that has been administered to a subject based on results of the monitoring step.

23. The method of claim 22, wherein the monitoring step comprises use of hyperpolarization magnetic resonance imaging.

24. The method of claim 23, wherein the metabolic pathway is a nucleotide synthesis pathway.

25. The method of claim 24, wherein the molecule is a carbon molecule.

26. The method of claim 25, wherein the carbon molecule becomes associated with one or more metabolites within the nucleotide synthesis pathway.

27. The method of claim 26, wherein the one or more metabolites are N-carbamoylaspartate, dihydroorotate, orotate, orotidine 5′-monophosphate (OMP), or uridine monophoshpate (UMP).

28. The method of claim 27, wherein quantifying the carbon molecule quantifies dihydroorotate or orotate levels.

29. The method of claim 27, further comprising determining a dose of the therapeutic agent based on dihydroorotate or orotate levels that is sufficient to inhibit an enzyme within the nucleotide synthesis pathway to an extent that at least one sign or symptom of the disorder is ameliorated, reduced, or eliminated.

30. The method of claim 29, wherein the method is repeated at a second point in time.

31. The method of claim 30, furthering comprising adjusting the dose of the therapeutic agent based on results of the method from the second point in time.

32. A device comprising a processor and a memory unit operably coupled to the processor to cause the processor to:
receive data that comprises a dose of a therapeutic agent and a time that a subject received the dose of the therapeutic agent, wherein the therapeutic agent inhibits a metabolic pathway of the subject;
generate a reminder that that provides a time when a next dose of the therapeutic agent should be administered to the subject, wherein the time when the next dose of the therapeutic agent should be administered is generated based on a relationship between the dose of the therapeutic agent and a threshold level of the metabolite, wherein administration of the next dose raises or maintains a level of the metabolite in the subject above the threshold level to sufficiently alter the metabolic pathway to thereby ameliorate, reduce, or eliminate at least one sign or symptom of a disorder in the subject; and
output the reminder to the subject.

33. The device of claim 32, wherein the reminder comprises at least one selected from the group consisting of an audible signal, a visual signal, a tactile signal, a vibration, and a combination thereof.

34. The device of claim 32, wherein the reminder is outputted to a component of the device.

35. The device of claim 32, wherein the reminder is outputted to a remote device.

36. The device of claim 32, wherein each of the time that a subject received the dose of the therapeutic agent and the time when the subject should administer the next dose of the therapeutic agent includes a date.

37. The device of claim 32, wherein the device stores information on doses of the therapeutic agent and time points when they were received by the subject.

38. The device of claim 37, wherein, based on the stored information, the processor:
determines whether intervals between time points in the stored information changes over time; and
determine that the subject has developed or is developing resistance to the therapeutic agent based on the intervals between the time points decreasing over time.

39. The device of claim 32, wherein the processor outputs a recommendation for adjusting a therapeutic course for the subject.

40. The device of claim 39, wherein the recommendation comprises administering a second therapeutic agent in addition to the therapeutic agent.

41. The device of claim 37, wherein the processor outputs the stored information on doses of the therapeutic agent and time points when they were received by the subject to a physician.

42. The device of claim 41, wherein the stored information enables the physician to:
determine that the subject has developed or is developing resistance to the therapeutic agent based on the intervals between the time points decreasing over time; and
adjust a therapeutic course for the subject.

43. A method for assessing in real-time an impact on a tumor of a therapeutic agent, the method comprising:
monitoring, in real time, an oxygenation level in a tumor; and
assessing an impact on the tumor of a therapeutic agent that has been administered to a subject based on results of the monitoring step.

44. The method of claim 43, wherein the monitoring step comprises use of electron paramagnetic resonance (EPR) imaging.

45. The method of claim 44, wherein the metabolic pathway is a nucleotide synthesis pathway.

46. The method of claim 43, wherein the agent is selected from the group consisting of: PALA (N-phosphoacetyl-L-aspartate), pyrazofurin, brequinar, a brequinar analog, a brequinar derivative, a brequinar prodrug, a micellar formulation of brequinar, and a brequinar salt.

47. The method of claim 46, wherein the brequinar salt is a sodium salt.

48. A method for assessing in real-time an impact on a tumor of a therapeutic agent, the method comprising:
monitoring, in real-time, a molecule that is associated with a metabolic pathway in a tumor as the molecule moves through the metabolic pathway in the tumor;
monitoring, in real time, an oxygenation level in a tumor; and
assessing an impact on the tumor of a therapeutic agent that has been administered to a subject based on results of both of the monitoring steps.

49. The method of claim 48, wherein monitoring the oxygenation level comprises use of electron paramagnetic resonance (EPR) imaging.

50. The method of claim 49, wherein monitoring the molecule comprises use of hyperpolarization magnetic resonance imaging.

51. The method of claim 48, wherein the metabolic pathway is a nucleotide synthesis pathway.

52. The method of claim 48, wherein the agent is selected from the group consisting of: PALA (N-phosphoacetyl-L-aspartate), pyrazofurin, brequinar, a brequinar analog, a brequinar derivative, a brequinar prodrug, a micellar formulation of brequinar, and a brequinar salt.

53. The method of claim 52, wherein the brequinar salt is a sodium salt.
